(19) United States
(12) Patent Application Publication
(10) Pub. No.: US 2024/0336647 A1
(43) Pub. Date: Oct. 10, 2024

(54) NUCLEOSIDE ANALOGS AND THERAPEUTIC USE THEREOF

(71) Applicant: Shiyue Fang, Houghton, MI (US)

(72) Inventor: Shiyue Fang, Houghton, MI (US)

(21) Appl. No.: 18/612,925

(22) Filed: Mar. 21, 2024

Related U.S. Application Data
(60) Provisional application No. 63/457,336, filed on Apr. 5, 2023.

(51) Int. Cl. C07H 19/207; A61K 31/7068; A61K 31/7076; C07H 19/10

(52) U.S. Cl. CPC C07H 19/207; A61K 31/7068; A61K 31/7076; C07H 19/10

(57) ABSTRACT

Nucleoside prodrugs featuring the naturally occurring modifications m1A and m3C, or their variants, are presented. These analogs function as therapeutics, by first integrating the modification into RNA or DNA during replication or by binding to RNA or DNA polymerase, and then, hindering the replication of synthesized DNA/RNA or inducing mutagenesis during subsequent replications. The nucleoside analogs can be used as antivirals, antibiotics, and anticancer agents as well as for other therapeutic applications. A distinction of these new therapeutics from many others is that their functional element is the naturally occurring m1A or m3C nucleobase modification.

### CROSS-REFERENCE TO RELATED APPLICATIONS

This application for patent claims priority to Provisional Application Ser. No. 63/457,336 filed on Apr. 5, 2023.

### FEDERALLY SPONSORED RESEARCH

This invention was made with United States government support under grant numbers GM109288 and 1954041 awarded by the National Institutes of Health and National Science Foundation, respectively. The government has certain rights in the invention.

### REFERENCE TO ELECTRONICALLY FILED SEQUENCE LISTING

The content of the electronically submitted Nucleotide Sequence Listing XML file (Name: RNA1.xml; Size: 18,888 bytes; Date of Creation: Jun. 9, 2024) is herein incorporated by reference in its entirety.

### BACKGROUND OF THE INVENTION

The present invention pertains to the field of therapeutic nucleoside analogs. Nucleoside analogs have garnered extensive utilization across a spectrum of applications [Hruba et al 2023 Biochem Pharmacol 215:115741 doi:10.1016/j.bcp.2023.115741; Ruiz et al 2022 Drug Discov Today 27:1832 doi:10.1016/j.drudis.2022.02.016; Borbone et al 2021 Molecules doi:10.3390/molecules26040986]. Human ailments, ranging from viral and bacterial infections to various cancers, have found remedy through drugs derived from such analogs. Nucleoside-based drugs operate through diverse functional mechanisms, one of which involves inhibiting DNA or RNA polymerase or inducing random mutagenesis. Upon polymerase inhibition, DNA or RNA replication, crucial for the proliferation of cancer cells or microorganisms, is halted, leading to potential disease resolution. Noteworthy examples encompass the antiviral drug remdesivir, alongside anticancer medications like cytarabine and fludarabine [Hruba et al 2023 Biochem Pharmacol 215:115741 doi:10.1016/j.bcp.2023.115741; Kokic et al 2021 Nat Commun 12:279 doi:10.1038/s41467-020-20542-0; Yin et al 2020 Science 368:1499 doi: 10.1126/science.abc1560; Clarke et al 2019 Pat No U.S. Pat. No. 10,251,904B2; Chun et al 2019 Pat No U.S. Pat. No. 10,251,898B2]. In certain scenarios, polymerase inhibition doesn't occur; rather, the drug induces lethal mutagenesis during DNA/RNA replication, exemplified by the antiviral drug favipiravir [Shannon et al 2020 Nat Commun 11:4682 doi:10.1038/s41467-020-18463-z].

While numerous nucleoside based drugs have been developed or described in the literature [Clarke et al 2019 Pat No U.S. Pat. No. 10,251,904B2; Chun et al 2019 Pat No U.S. Pat. No. 10,251,898B2; Kalman 2020 Pat No U.S. Pat. No. 10,751,358B2; Kalman 2023 Pat No U.S. Pat. No. 11,618,765B2; Chang et al 2015 Pat No U.S. Pat. No. 9,095,599B2; Wagner 2018 Pat Appl No US2018/0258131A1; McGuigan et al 2006 Pat Appl No WO2006/100439A1], the exploration of the structural space of nucleoside analogs remains far from exhaustive. In particular, modification of the N1 position of adenine and N3 position of cytosine nucleobases for therapeutic development has not received attention. Reasons behind this may include concerns of lack of bioactivity due to complete disruption of Watson Crick hydrogen bonding and unconscious inclination to modify by replacing an atom or a group of atoms of natural nucleosides with something than by replacing an electron lone pair. However, delving into the realm of modifying these unexplored positions may offer opportunities to address the limitations of existing medications, including toxicity, the need for early administration for treating infectious diseases, adverse effects, drug resistance, and inefficacy in certain patient populations. Notably, none of the nucleoside-based drugs currently approved by the Food and Drug Administration (FDA) for Coronavirus Disease 2019 (COVID-19) can be deemed ideal. Remdesivir, for instance, may lead to adverse effects such as rapid heartbeat, respiratory issues, coughing, headaches, among others [Sedighi et al 2022 J Med Virol 94:3783 doi:10.1002/jmv.27800]. Similarly, molnupiravir is associated with side effects like vomiting, dizziness, and diarrhea [Kauer et al 2023 Viruses doi:10.3390/v15051181]. Given these considerations, the development of new nucleoside drugs based on modifications at the N1 position of adenine and N3 position of cytosine nucleobases is a significant endeavor. These new drugs may be able to address problems that cannot be addressed using existing drugs.

### BRIEF SUMMARY OF THE INVENTION

Irrelevant to the goal of developing new drugs, but purely motivated by a curiosity to unravel the intricacies behind the personalized manifestation of symptoms during the COVID-19 pandemic, we embarked on a study on the effects of epitranscriptomic RNA modifications, including methylations, on the catalytic activity of the RNA-dependent RNA polymerase (RdRp) of the severe acute respiratory syndrome coronavirus 2 (SARS-COV-2). Among other findings, our investigation revealed that the epitranscriptomic modification N1-methyladenosine (m1A) [F001] leads to inhibition of the enzyme, while N3-methylcytidine (m3C) [F002] has minimal impact, when the modifications are present in template RNA [Apostle et al 2023 ChemBioChem doi:10.1002/cbic.202300095]. Surprisingly, both m1A and m3C disrupt the canonical Watson-Crick hydrogen bonding in the same manner, yet exhibit vastly contrasting effects on RdRp activity.

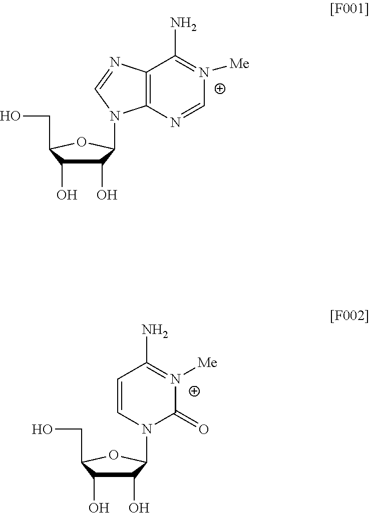

The above discoveries prompted us to explore the possibility of developing new drugs by modifying the N1 position of adenine and N3 position of cytosine. The hypothesis is that if m1A or m3C modification can be introduced onto an RNA or DNA through therapeutic means, the modified RNA or DNA would not be replicable or cause lethal mutagenesis (due to their disruption of Watson Crick hydrogen bonding) in subsequent replication cycles. Two general methods could be considered to introduce m1A or m3C. One is to tune the epitranscriptomic and epigenetic machinery to increase cellular level or activity of methylase or decrease cellular level or activity of demethylase. The other is to deliver prodrugs that can be converted to m1ATP [F003] or m3CTP in vivo to incorporate m1A or m3C into RNA or DNA by cellular or viral polymerases. The later is simpler and could go beyond methylation such ethylation. However, considering that m1A in template RNA was intolerable by SARS-COV-2 RdRp [Apostle et al 2023 ChemBioChem doi:10.1002/cbic.202300095], incorporation of m1A into RNA by polymerase via m1ATP was perceived difficult. Nonetheless, owning to the potentially high impact and ease of the needed experiments, we explored this possibility any way, and were surprised to discover that m1A could indeed be efficiently incorporated into RNA by SARS-COV-2 RdRp [Apostle et al Preprints doi:10.20944/preprints202311.1268.v1].

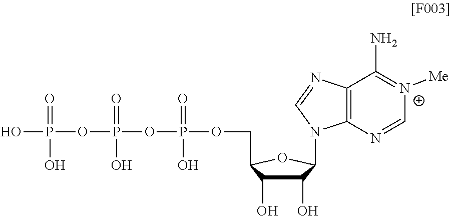

Therefore, provided in this invention are nucleoside analogs including their pharmaceutically acceptable salts, hydrates, esters, amides, or other formulations based on m1A and m3C, and their use as antiviral and anticancer therapeutics via mechanisms including but are not limited to inhibition of DNA or RNA polymerases, or causing lethal mutagenesis. Both m1A and m3C carry a positive charge under physiological pH due to their methylation modification. The methyl group introduced by the modifications disrupts Watson Crick hydrogen bonding in a similar manner. Consequently, their interactions with DNA or RNA polymerases, as well as other cellular components, are expected to share a high degree of similarity. The nucleoside analogs can be represented by [F004] and [F005]:

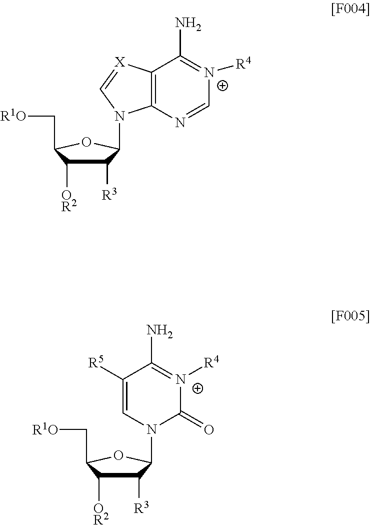

wherein R¹ is a group that does not hinder the compounds from passing biological membranes and can be converted to a triphosphate under physiological conditions after passing biological membranes. R² is H, Me, Et, or groups that can be converted to H under physiological conditions. R³ is H, F, O(CH₂)₂OH, O(CH₂)₂OMe, O(CH₂)₂NH₂, O(CH₂)₂NHMe, O(CH₂)₂NMe₂, ONH₂, or OR². R⁴ is Me or Et. R⁵ is H or Me. X is N or CH. More detailed definition of the variable atoms and groups is provided in the Detailed Description of the Invention and Claims sections.

Compared with known nucleoside based prodrugs for treating human diseases including infectious diseases and various cancers [Clarke et al 2019 Pat No U.S. Pat. No. 10,251,904B2; Chun et al 2019 Pat No U.S. Pat. No. 10,251,898B2; Kalman 2020 Pat No U.S. Pat. No. 10,751,358B2; Kalman 2023 Pat No U.S. Pat. No. 11,618,765B2; Chang et al 2015 Pat No U.S. Pat. No. 9,095,599B2; Wagner 2018 Pat Appl No US2018/0258131A1; McGuigan et al 2006 Pat Appl No WO2006/100439A1], the prodrugs based on [F004] and [F005] have the distinction of being based on the naturally occurring m1A and m3C modifications. In the majority of the embodiments of the invention, the ultimate functional element the drugs bring about to biological systems is m1A or m3C on RNA or DNA. These elements have been found in normal biological systems, and are related to human diseases [Song et al 2023 Genes Dis 10:739 doi:10.1016/j.gendis.2022.11.001; Shafik et al 2022 Hum Mol Genet 31:1673 doi:10.1093/hmg/ddab357; Ignatova et al 2020 Sci Adv doi:10.1126/sciadv.aaz4551]. They may be one of the reasons for the resistance to certain infectious diseases and for lower risk to cancer by some people. Therefore, the functional mechanism of the drugs of some embodiments of this invention can be considered as tuning the epitranscriptomic or epigenetic machinery of patients or more vulnerable population to match that of healthy population. As a result, these new drugs could be less toxic than known nucleoside drugs.

### DEFINITIONS

Alkenyl is a hydrocarbon containing at least one carbon-carbon double bond, and lacking one hydrogen atom. Examples include but are not limited to —CH═CH₂, and —CH₂CH═CH₂. Allyl is —CH₂CH═CH₂.

Alkoxy is a group with the formula —O-alkyl. Examples include but are not limited to —OMe.

Alkyl is hydrocarbon containing normal, secondary and tertiary or cyclic carbon atoms, and lacking one hydrogen atom. Examples include but are not limited to —CH₃.

Alkynyl is a hydrocarbon containing at least one carbon-carbon triple bond, and lacking one hydrogen atom. Examples include but are not limited to —C═CH, and —CH₂C═CH.

Aryl is an aromatic hydrocarbon lacking one hydrogen atom. Examples include but are not limited to phenyl.

Benzyl is —CH₂Ph.

Carbocycle refers to saturated, partially unsaturated or aromatic carbon ring. It includes monocycle, bicycle, and polycycle.

Carbocyclyl is a carbocycle lacking one hydrogen atom.

Carbocyclylalkyl refers to an acyclic alkyl group with one of its hydrogen being replaced by a carbocyclyl.

Compounds in this application, depending on the context they appear, may include their protonated and deprotonated forms, different tautomers, simple esters and amides that can be converted to the indicated compounds under physiological conditions predictable by persons skilled in the art.

Et is ethyl group (—CH₂CH₃).

Formula of a molecule in this application should not be construed as limiting the scope of the molecule. The molecule represented by the formula, depending on the context they appear, may include their protonated and deprotonated forms; different tautomers; different forms of salts, esters and amides; and those containing unusual one or more isotopes of atoms.

Heteroaryl refers to an aryl having at least one heteroatom in the ring of the aryl.

Heteroalkyl is an alkyl group with one or more carbon atoms being replaced by a heteroatom such as O, N, S or halogen. Examples include but are not limited to —CH₂OMe.

Heteroarylalkyl is an alkyl group with at least one hydrogen being replaced by heteroaryl group.

Heterocycle is a carbon cycle with at least one carbon forming the cycle being replaced by a heteroatom such as O, N, or S.

Heterocyclyl is a heterocycle lacking one hydrogen atom. Examples include but are not limited to —N(CH₂CH₂)₂S.

Isoquinolinyl is isoquinoline lacking one hydrogen atom.

m1A, depending on the context it appears, may refer to N1-methyladenosine, N1-methyldeoxyadenosine, N1-methyladenine, or their nucleotide mono-, bi-, and tri-phosphates. m1ATP is triphosphate of m1A.

m3C, depending on the context it appears, may refer to N3-methylcytidine, N3-methyldeoxycytidine, N3-methylcytosine, or their nucleotide mono-, bi-, and tri-phosphates, as well as their 5-methylated version.

m3CTP is triphosphate of m3C.

Me is methyl group (—CH₃).

Naphthyl is naphthalene lacking a hydrogen atom.

Nucleotide prodrug is a nucleoside derivative that does not contain any negatively charged phosphate group of nucleotides, and thus can pass the negatively charged biological membranes; but once in the cell, can be converted to nucleotide monophosphate, biphosphate, and triphosphate by cellular enzymes, and become the active drug.

Optionally substituted in reference to a particular moiety of a compound means that the hydrogen atoms of the moiety may or may not be replaced by one or more non-hydrogen atoms or groups of atoms.

Optionally replaced in reference to a particular moiety of a compound means that the moiety may or may not been replaced by an atom or groups of atoms.

Phenyl is —C₆H₅ or —Ph.

Propargyl is —CH₂C═CH.

Quinolinyl is quinoline lacking a hydrogen atom.

Substituted in reference to alkyl, alkenyl, aryl, arylalkyl, alkoxy, heterocyclyl, heteroaryl, carbocyclyl, etc. means that one or more hydrogen atoms of the associated groups are each independently replaced by a non-hydrogen atom or group of atoms.

### BRIEF DESCRIPTION OF DRAWINGS

FIG. 1. Gel images for determining if m1A and m3C in template RNA could inhibit SARS-CoV-2 RdRp.

FIG. 2. Gel image for determining if m1A could be incorporated onto RNA by SARS-COV-2 RdRp via m1ATP.

FIG. 3. Conversion of an m1A prodrug to its bioactive form.

FIG. 4. Synthesis of an m3C phosphoramidite.

FIG. 5. Synthesis of a phosphoramidate prodrug of m1A.

FIG. 6. Synthesis of an ester prodrug of m1A.

FIG. 7. RNA sequences for determining if m1A and m3C in template RNA can inhibit RdRp.

FIG. 8. RNA sequences for determining m3C mutagenesis.

### DETAILED DESCRIPTION OF THE INVENTION

During the COVID-19 pandemic, there was considerable interest in understanding why some individuals experience more severe symptoms from the same virus compared to others. Apart from the established factors outlined in existing literature, such as induced immunity [McCoy et al 2020 Vaccines doi:10.3390/vaccines8040700], we hypothesized that differences in individuals' epitranscriptomic systems could also contribute. For example, variations in epitranscriptomic enzymes might affect the methylation or demethylation of viral RNA genomes, thereby influencing viral propagation.

Building on the above hypothesis, we investigated the impact of known RNA modifications, including methylation, on the catalytic activity of SARS-COV-2 RdRp [Apostle et al 2023 ChemBioChem doi:10.1002/cbic.202300095]. RdRp is crucial for both replicating the viral RNA genome and transcribing viral mRNAs, making its activity pivotal for viral propagation. We discovered that the m1A modification [F001], when it is in the template RNA, inhibits SARS-COV-2 RdRp. Thereby, it is possible that individuals whose epitranscriptomic system is more capable of installing or less capable of erasing the m1A modification in viral RNA genome could be less vulnerable to COVID-19. We also discovered that the m3C modification [F002], when it is in the template RNA, has little effect on the RdRp. Although m3C does not inhibit RdRp, because of the disruption of Watson Crick base pairing by the methyl group, this modification, if installed on a viral genome, would cause mutagenesis, and therefore would attenuate viral propagation. Thereby, similar to the m1A modification, it is possible that individuals whose epitranscriptomic system is more capable of installing or less capable of erasing the m3C modification in viral RNA genome could be less vulnerable to COVID-19. Despite the fact that both m1A and m3C, if installed on viral RNA genome by an individual's epitranscriptomic system, would make the individual less vulnerable to COVID-19, the discoveries, the former inhibits the enzyme while the latter has little effect, are surprising because both m1A and m3C disrupt canonical base pairing in a similar manner, both impose similar steric hindrance in the catalytic pocket of RdRp, and both have a positive charge under physiological pH generated by the methylation.

Prompted by the above discoveries and rationales, we reasoned that if the m1A or m3C modification could be introduced onto viral RNA genome by RdRp via m1ATP or m3CTP, respectively, during viral genome replication, novel antiviral therapeutics based on m1A or m3C could be designed and developed. The functional mechanism of these new drugs would involve four steps. (1) A nucleoside analog containing m1A or m3C modification in the form of nucleotide prodrug passes various biological membranes and enters target cells. (2) In the cells, the nucleoside analog undergoes enzymatic reactions to give m1ATP or m3CTP. (3) m1ATP or m3CTP participates in the RNA genome replication as well as mRNA transcription reaction catalyzed by RdRp, installing multiple m1A or m3C modifications onto the progeny RNA genome. (4) The m1A or m3C modifications on the progeny RNA genome stops further propagation of the virus via stopping further replication of viral genome in the case of m1A, or via causing lethal mutagenesis during the next generation genome replication in the case of m3C. In addition, there is also a possibility that m1ATP or m3CTP or a molecule derived from them inhibits the polymerase.

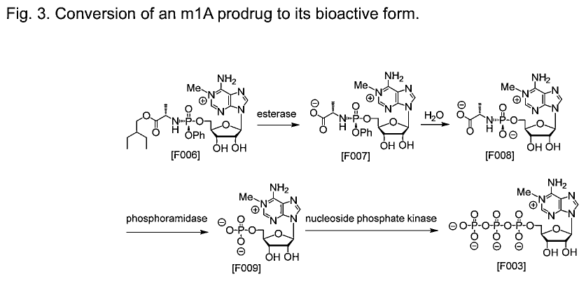

A specific example for the functional process, without limiting the scope of the invention, is given in FIG. 3. The m1A nucleoside analog [F006] does not have a negative charge and should be able to pass the negatively charged biological membranes. Once in a cell, esterase will convert it to [F007], which will undergo hydrolysis to give [F008]. [F008] will be converted to the m1A monophosphate [F009] by phosphoramidase. Phosphorylation by nucleoside phosphate kinase will give the m1ATP, which can participate in the RdRp catalyzed viral genome replication reaction to give m1A decorated viral genome, preventing further replication of the viral genome and viral propagation.

Despite the potential breakthrough and enormous benefits in developing a new class of therapeutics if the m1A or m3C modification could be installed onto RNA by RdRp via m1ATP or m3CTP, respectively, it had been understood that the likelihood of success for the m1A or m3C installation were extremely low if not impossible. The reason was that the methyl group introduced by the modifications poses significant steric hindrance in a critical space that is filled with only an electron lone pair in the case the incorporation of standard unmodified A or C via ATP or CTP. Even more of a concern was that m1A in the template RNA had been found to inhibit the RdRp as described above, which could typically be interpreted as intolerance of m1A by RdRp.

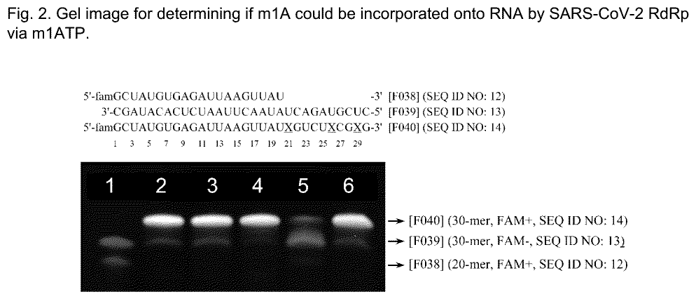

However, after carrying out the studies involving m1A and m3C in the template RNA, we had everything in our lab for the experiments needed to determine if m1A could be installed onto RNA by RdRp. The only exception was m1ATP, which, fortunately, was commercially available. Therefore, despite the perceived low chance of success, we incubated m1ATP and other nucleoside triphosphates (NTPs) with an RNA primer-template complex in the presence of RdRp. Results are given in FIG. 2, and detailed procedure is given in the Example Procedure section. Surprisingly, m1A can be incorporated onto RNA, in spite of the observed intolerance of m1A by RdRp when it is in template RNA [Apostle et al Preprints doi:10.20944/preprints202311.1268.v1].

With the aforementioned discoveries and straightforward rationales, there is a reasonable chance of success for the following. The m3C, which was found tolerable by RdRp when it was in template RNA, could also be installed onto RNA by RdRp via m3CTP. Besides SARS-COV-2 RdRp, RdRp of other viruses have a reasonable chance to behave similarly. Further, there is also a reasonable chance that other polymerases including RNA dependent DNA polymerases, DNA dependent RNA polymerases, and DNA dependent DNA polymerases in other microorganisms as well as in cancer cells could behave similarly toward the m1A and m3C modifications involving RNA or DNA.

Therefore, provided, are nucleoside analogs of structural Formulas [F010] and [F011]

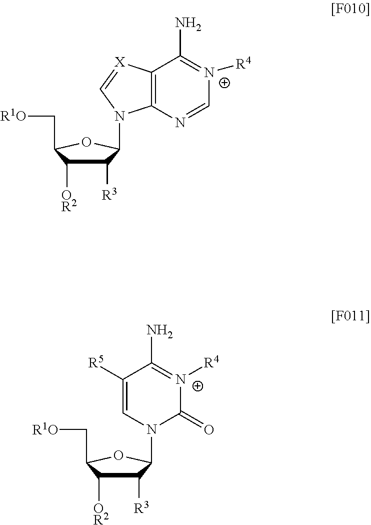

or a pharmaceutically acceptable salt, hydrate, ester, amide, or formulation thereof, wherein, R¹ is selected from the group consisting of items in subgroups (a-c):
(a) —(C═O)R^{1a},
   wherein R^{1a} is (C₃-C₂₀)alkyl, (C₂-C₂₀)alkenyl, (C₂-C₂₀)alkynyl, (C₄-C₈) carbocyclylalkyl, (C₆-C₂₀) optionally substituted aryl, (C₆-C₂₀) optionally substituted heteroaryl, or (C₆-C₂₀)aryl(C₁-C₈)alkyl;
(b) —(C═O)OR^{1b}, —C(C═O)NR^{1b}R^{1c}, —C(C═O)SR^{1b}, —S(O)R^{1b}, —S(O)₂R^{1b}, —S(O)(OR^{1b}), —S(O)₂(OR^{1b}), and —SO₂NR^{1b}R^{1c};
   wherein each R^{1b} and R^{1c} is independently H, (C₁-C₂₀)alkyl, (C₂-C₂₀)alkenyl, (C₂-C₂₀)alkynyl, (C₄-C₈)carbocyclylalkyl, (C₆-C₂₀) optionally substituted aryl, (C₆-C₂₀) optionally substituted heteroaryl, or (C₆-C₂₀)aryl (C₁-C₈)alkyl; or R^{1b} and R^{1c} taken together with a nitrogen to which they are both attached form a 3 to 7 membered heterocyclic ring wherein any one carbon atom of said heterocyclic ring can optionally be replaced with —O—, —S— or —NR^{1d}; wherein
   each R^{1d} is independently selected from the group consisting of H, (C₁-C₂₀)alkyl, (C₂-C₂₀)alkenyl, (C₂-C₂₀)alkynyl, (C₆-C₂₀)aryl, (C₆-C₂₀)aryl (C₁-C₈)alkyl, (C₄-C₈)carbocyclylalkyl, —C(═O) R^{1e}, —C(═O)OR^{1e}, —C(═O)NR^{1e}₂, —C(═O)SR^{1e}, —S(O)R^{1e}, —S(O)₂R^{1e}, —S(O)(OR^{1e}), —S(O)₂(OR^{1e}), and —SO₂NR^{1e}₂; wherein
   each R^{1e} is independently selected from the group consisting of H, (C₁-C₂₀)alkyl, (C₁-C₂₀) substituted alkyl, (C₂-C₂₀)alkenyl, (C₂-C₂₀) substituted alkenyl, (C₂-C₂₀)alkynyl, (C₂-C₂₀) substituted alkynyl, (C₆-C₂₀)aryl, (C₂-C₂₀) substituted aryl, (C₂-C₂₀)heterocyclyl, (C₂-C₂₀) substituted heterocyclyl, (C₆-C₂₀)aryl (C₁-C₈)alkyl, and substituted (C₆-C₂₀)aryl (C₁-C₈)alkyl;
   wherein each (C₁-C₂₀)alkyl, (C₂-C₂₀)alkenyl, (C₂-C₂₀)alkynyl, and (C₆-C₂₀)aryl (C₁-C₈)alkyl of each R^{1a}, R^{1b}, R^{1c}, R^{1d} and R^{1e} is, independently, optionally substituted with one or more substituents selected from the group consisting of halo, hydroxy, CN, N₃, N(R^{1d})₂ and OR^{1d}; and wherein one or more of the non-terminal carbon atoms of each said (C₁-C₂₀)alkyl is optionally replaced with —O—, —S—, or —NR^{1d}—;
and
(c) Formulas [F012], [F013] and [F014]

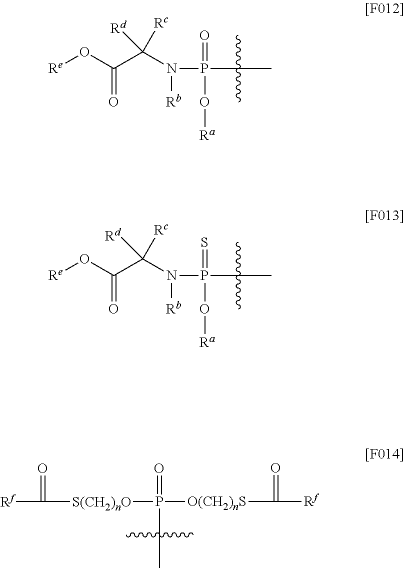

wherein
   R^{a} is selected from the group consisting of phenyl, 1-naphthyl, 2-naphtyl, quinolinyl, and isoquinolinyl;
   R^{b} is selected from the group consisting of H, Me, —(CH₂)₂OMe, —(CH₂)₂O-substituted Me, —(CH₂)₂OPh, and —(CH₂)₂O-substituted Ph;
   R^{c} and R^{d} are each independently selected from the group consisting of H, (C₁-C₆)alkyl, allyl, propargyl, and benzyl;
   R^{e} is selected from the group consisting of H, (C₁-C₈)alkyl, benzyl, allyl, propargyl, (C₃-C₆)cycloalkyl, and —CH₂—(C₃-C₆)cycloalkyl;
   R^{f} is selected from the group consisting of (C₁-C₈)alkyl,-O-(C₁-Cₛ)alkyl, benzyl, —O-benzyl, allyl, —O-allyl, propargyl, —O-propargyl, —CH₂—(C₃-C₆)cycloalkyl, —O—CH₂—(C₃-C₆)cycloalkyl, and CF₃; and
   n is an integer selected from the group consisting of 1, 2, 3, and 4;

R² is selected from the group consisting of H, Me, —(C═O)R^{1b}, —(C═O)OR^{1b}, —C(C═O)NR^{1b}R^{1c}, —C(C═O)SR^{1b}, —S(O)R^{1b}, —S(O)₂R^{1b}, —S(O)(OR^{1b}), —S(O)₂(OR^{1b}), and —SO₂NR^{1b}R^{1c};

R³ is selected from the group consisting of H, F, O(CH₂)₂OH, O(CH₂)₂OMe, O(CH₂)₂NH₂, O(CH₂)₂NHMe, O(CH₂)₂NMe₂, ONH₂, and OR₂;

R⁴ is selected from the group consisting of Me and Et;

R⁵ is selected from the group consisting of H and Me; and

X is selected from the group consisting of N, and CH.

Some of the embodiments consist of the compounds of Formulas [F010] and [F011] or their pharmaceutically acceptable salts, esters, amide, or formulations, wherein R¹ is —(C═O)R^{1a} with R^{1a} being (C₃-C₂₀)alkyl.

Some of the embodiments consist of the compounds of Formulas [F010] and [F011] or their pharmaceutically acceptable salts, esters, amide, or formulations, wherein said R¹ is —(C═O)OR^{1b} with R^{1b} being (C₁-C₂₀)alkyl.

Some of the embodiments consist of the compounds of Formulas [F010] and [F011] or their pharmaceutically acceptable salts, esters, amide, or formulations, wherein said R¹ is selected from the group consisting of Formulas [F012], [F013] and [F014].

Some of the embodiments consist of the compounds or their pharmaceutically acceptable salts, esters, amide, or formulations selected from the group consisting of

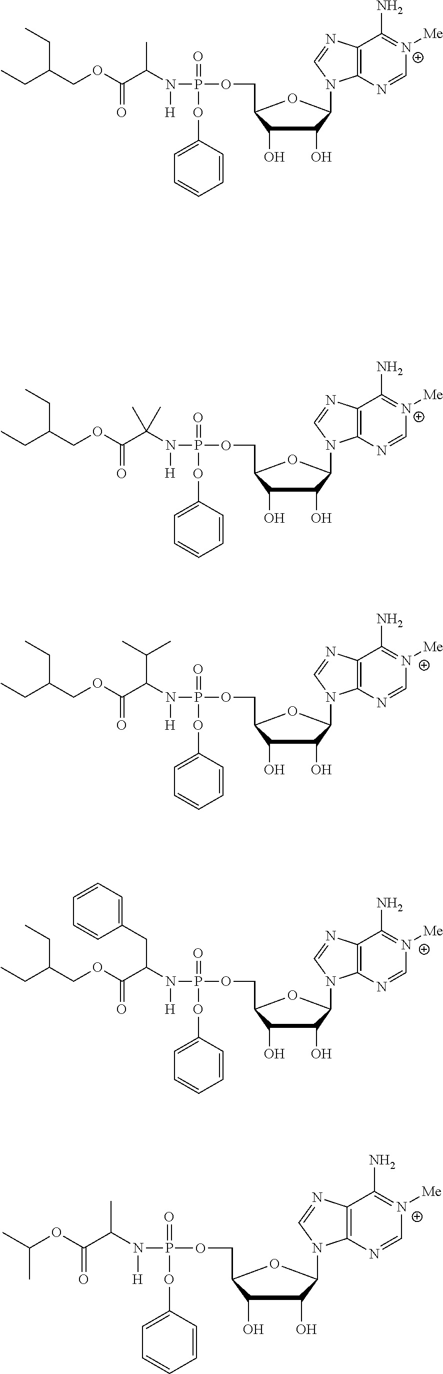

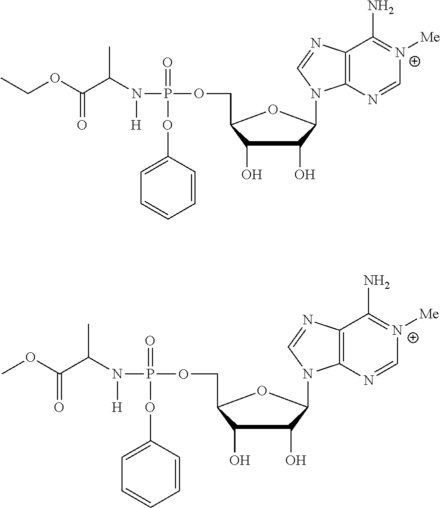

Some of the embodiments consist of the compounds or their pharmaceutically acceptable salts, esters, amide, or formulations selected from the group consisting of

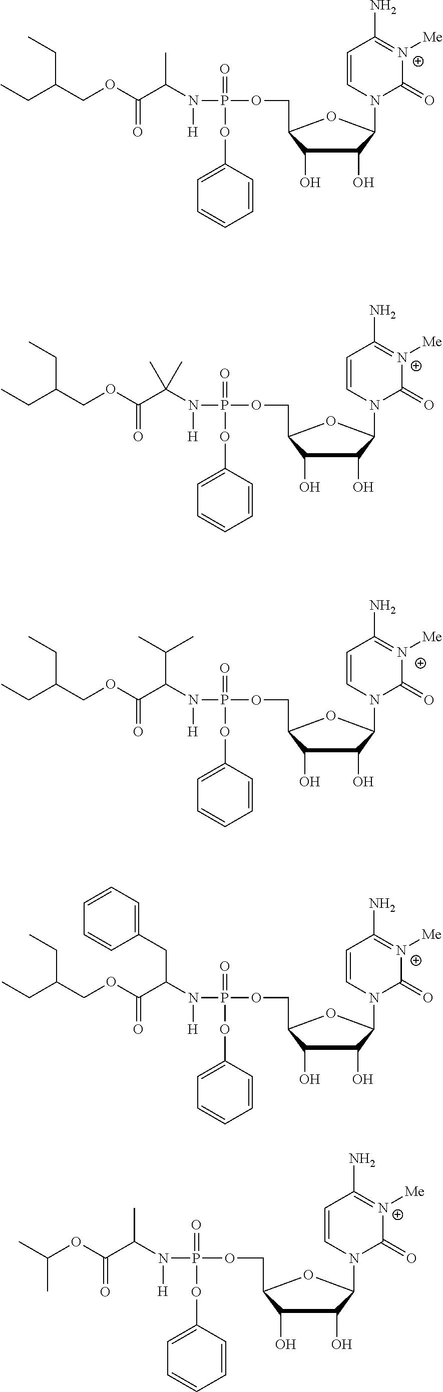

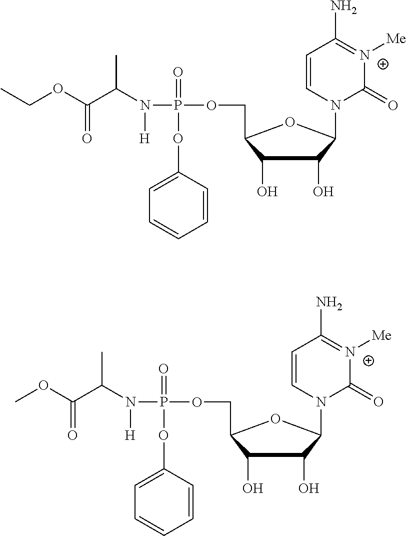

Also, provided, are methods of preventing and treating cancer and infectious disease in a human involving the use of a compound with Formula [F010] or [F011]

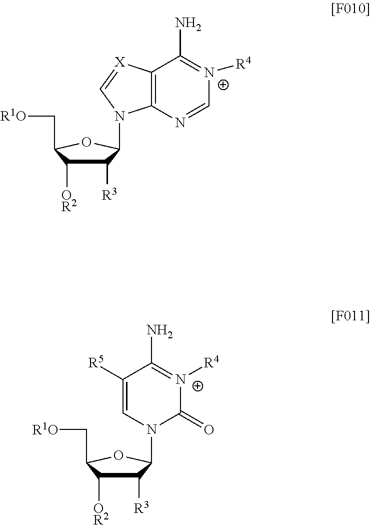

or a pharmaceutically acceptable salt, hydrate, ester, amide, or formulation thereof, wherein, R¹ is selected from the group consisting of subgroups (a-c):
(a) —(C═O)R^{1a},
   wherein R^{1a} is (C₃-C₂₀)alkyl, (C₂-C₂₀)alkenyl, (C₂-C₂₀)alkynyl, (C₄-C₈)carbocyclylalkyl, (C₆-C₂₀) optionally substituted aryl, (C₆-C₂₀) optionally substituted heteroaryl, or (C₆-C₂₀)aryl (C₁-C₈)alkyl;
(b) —(C═O)OR^{1b}, —C(C═O)NR^{1b}R^{1c}, —C(C═O)SR^{1b}, —S(O)R^{1b}, —S(O)₂R^{1b}, —S(O)(OR^{1b}), —S(O)₂(OR^{1b}), and —SO₂NR^{1b}R^{1c};
   wherein each R^{1b} and R^{1c} is independently H, (C₁-C₂₀)alkyl, (C₂-C₂₀)alkenyl, (C₂-C₂₀)alkynyl, (C₄-C₈)carbocyclylalkyl, (C₆-C₂₀) optionally substituted aryl, (C₆-C₂₀) optionally substituted heteroaryl, or (C₆-C₂₀)aryl (C₁-C₈)alkyl; or R^{1b} and R^{1c} taken together with a nitrogen to which they are both attached form a 3 to 7 membered heterocyclic ring wherein any one carbon atom of said heterocyclic ring can optionally be replaced with —O—, —S— or —NR^{1d}; wherein
   each R^{1d} is independently selected from the group consisting of H, (C₁-C₂₀)alkyl, (C₂-C₂₀)alkenyl, (C₂-C₂₀)alkynyl, (C₆-C₂₀)aryl, (C₆-C₂₀)aryl (C₁-C₈)alkyl, (C₄-C₈)carbocyclylalkyl, —C(═O)R^{1e}, —C(═O)OR^{1e}, —C(═O)NR^{1e}₂, —C(═O)SR^{1e}, —S(O)R^{1e}, —S(O)₂R^{1e}, —S(O)(OR^{1e}), —S(O)₂(OR^{1e}), and —SO₂NR^{1e}₂; wherein
   each R^{1e} is independently selected from the group consisting of H, (C₁-C₂₀)alkyl, (C₁-C₂₀) substituted alkyl, (C₂-C₂₀)alkenyl, (C₂-C₂₀) substituted alkenyl, (C₂-C₂₀)alkynyl, (C₂-C₂₀) substituted alkynyl, (C₆-C₂₀)aryl, (C₂-C₂₀) substituted aryl, (C₂-C₂₀)heterocyclyl, (C₂-C₂₀) substituted heterocyclyl, (C₆-C₂₀)aryl (C₁-C₈)alkyl, and substituted (C₆-C₂₀)aryl (C₁-C₈)alkyl;
   wherein each (C₁-C₂₀)alkyl, (C₂-C₂₀)alkenyl, (C₂-C₂₀)alkynyl, and (C₆-C₂₀)aryl (C₁-C₈)alkyl of each R^{1a}, R^{1b}, R^{1c}, R^{1d} and R^{1e} is, independently, optionally substituted with one or more substituents selected from the group consisting of halo, hydroxy, CN, N₃, N(R^{1d})₂ and OR^{1d}; and wherein one or more of the non-terminal carbon atoms of each said (C₁-C₂₀)alkyl is optionally replaced with —O—, —S—, or —NR^{1d}—;
and
(c) Formulas [F012], [F013] and [F014]

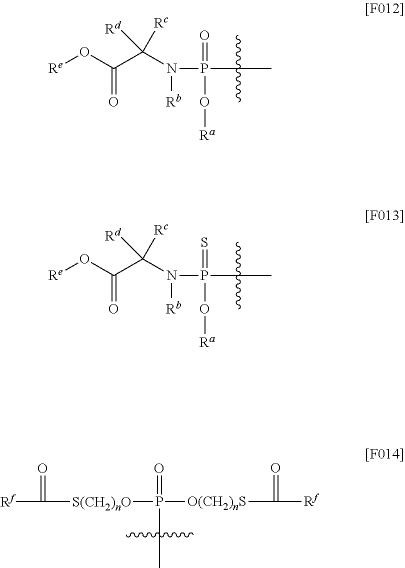

wherein
   R^{a} is selected from the group consisting of phenyl, 1-naphthyl, 2-naphtyl, quinolinyl, and isoquinolinyl;
   R^{b} is selected from the group consisting of H, Me, —(CH₂)₂OMe, —(CH₂)₂O-substituted Me, —(CH₂)₂OPh, and —(CH₂)₂O-substituted Ph;
   R^{c} and R^{d} are each independently selected from the group consisting of H, (C₁-C₆)alkyl, allyl, propargyl, and benzyl;
   R^{e} is selected from the group consisting of H, (C₁-C₈)alkyl, benzyl, allyl, propargyl, (C₃-C₆)cycloalkyl, and —CH₂—(C₃-C₆)cycloalkyl;
   R^{f} is selected from the group consisting of (C₁-C₈)alkyl, —O—(C₁-C₈)alkyl, benzyl, —O-benzyl, allyl, —O-allyl, propargyl, —O-propargyl, —CH₂—(C₃-C₆) cycloalkyl, —O—CH₂—(C₃-C₆)cycloalkyl, and CF₃; and
   n is an integer selected from the group consisting of 1, 2, 3, and 4;

R² is selected from the group consisting of H, Me, —(C═O)R^{1b}, —(C═O)OR^{1b}, —C(C═O)NR^{1b}R^{1c}, —C(C═O)SR^{1b}, —S(O)R^{1b}, —S(O)₂R^{1b}, —S(O)(OR^{1b}), —S(O)₂(OR^{1b}), and —SO₂NR^{1b}R^{1c};

R³ is selected from the group consisting of H, F, O(CH₂)₂OH, O(CH₂)₂OMe, O(CH₂)₂NH₂, O(CH₂)₂NHMe, O(CH₂)₂NMe₂, ONH₂, and OR²;

R⁴ is selected from the group consisting of Me and Et;

R⁵ is selected from the group consisting of H and Me; and

X is selected from the group consisting of N, and CH.

Some of the embodiments are methods of preventing or treating cancer or infectious disease in a human involving the use of a compound selected from the group consisting of

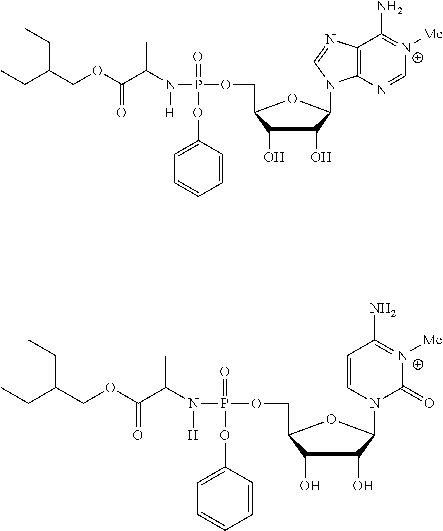

Some of the embodiments are methods of preventing or treating cancer.

Some of the embodiments are methods of preventing or treating infectious disease caused by severe acute respiratory syndrome coronavirus 2, and severe acute respiratory syndrome coronavirus.

Some of the embodiments are methods of preventing or treating infectious disease caused by human immunodeficiency virus.

Some of the embodiments are methods of preventing or treating infectious disease caused by filoviruses.

Some of the embodiments are methods of preventing or treating infectious disease caused by influenza viruses.

Some of the embodiments are methods of preventing or treating infectious disease caused by common cold viruses.

Some of the embodiments are methods of preventing or treating infectious disease caused by hepatitis C viruses.

Some of the embodiments are methods of preventing or treating human diseases involving the use of more than one compound derived from the m1A and m3C modifications as well as the use of one or more compounds derived from the m1A and m3C modifications in combination with other therapeutic agents. One example other therapeutic agent is compounds capable of inhibiting enzymes that can remove the methyl group from m1A or m3C. Another example other therapeutic agent is known antiviral agents for the treatment of HIV infection.

### EXAMPLE PROCEDURES

### Compound Synthesis

All compounds in this invention can be synthesized by persons skilled in the art without excessive experimentations. The descriptions or examples provided herein are intended for illustrative purpose only and should not be construed as limiting the scope of the invention.

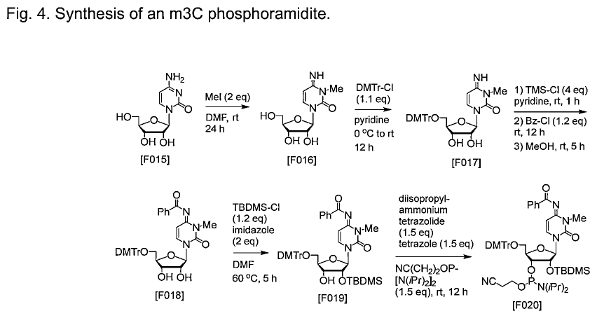

For conducting enzymatic assays to determine if the m1A or m3C modification in template RNA or DNA could inhibit a RNA or DNA polymerase, or cause mutagenesis, an RNA or DNA template containing the modification is needed. To synthesize the template RNA or DNA, a phosphoramidite monomer is needed. The synthesis of the phosphoramidite [F020], which is needed for the synthesis of RNA template containing the m3C modification, is given as an example. FIG. 4 provides the synthesis Scheme.

Synthesis of [F016] (FIG. 4): lodomethane (5.12 mL, 82.2 mmol) was added to the solution of [F015] (10 g, 41.1 mmol) in 100 mL DMF. The mixture was stirred at rt for 24 h. Volatiles were evaporated under vacuum from an oil pump. The residue was co-evaporated with toluene twice, and dissolved in acetone (20 mL). Hexane (50 mL) was added. The mixture was sonicated, and then kept at −20° C. overnight. The precipitate was filtered and washed with a cold mixture of acetone and hexanes (1:1 v/v, 50 mL×2). The solid was dried under vacuum to give [F016] as light yellow solid in 14.90 g, 94% yield. TLC R_{f}=0.2 (SiO₂, DCM/EtOAc/MeOH 2:2:3). ¹H and ¹³C NMR are consistent with reported data [Mao et al 2021 ACS Chem Biol 16:76 doi:10.1021/acschembio.0c00735; Moreno et al 2022 Monatsh Chem 153:285 doi:10.1007/s00706-022-02896-x].

Synthesis of [F017] (FIG. 4): Compound [F016] (7.0 g, 27.2 mmol) in pyridine (100 mL) was cooled on an ice bath. DMTr-CI (10.14 g, 29.9 mmol) in pyridine (50 mL) was added dropwise via a cannula. The reaction was allowed to proceed at rt overnight. Volatiles were evaporated under vacuum. The residue was partitioned between DCM (150 mL) and Na₂CO₃ (5%, 150 mL×2). The organic phase was dried over anhydrous Na₂SO₄ and evaporated under reduced pressure. Flash column chromatography (SiO₂, DCM/EtOAc/TEA 1:1:0.08 with gradually increased MeOH) gave [F017] as a white foam in 8.56 g, 56% yield. TLC R_{f}=0.3 (SiO₂, DCM/EtOAc/MeOH/TEA 1:1:0.08:0.08). ¹H and ¹³C NMR are consistent with reported data [Mao et al 2021 ACS Chem Biol 16:76 doi:10.1021/acschembio.0c00735; Moreno et al 2022 Monatsh Chem 153:285 doi:10.1007/s00706-022-02896-x].

Synthesis of [F018] (FIG. 4): Chlorotrimethylsilane (3.6 mL, 28.5 mmol) was added to a solution of [F017] (4 g, 7.14 mmol) in pyridine (80 mL). After stirring at rt for 1 h, benzoyl chloride (1.0 mL, 8.57 mmol) was added. The mixture was stirred at rt overnight, and then cooled to 0° C. Methanol (21.7 mL, 538.3 mmol) was added, and the mixture was stirred at rt for 5 h. Volatiles were evaporated under reduced pressure. Flash column chromatography (SiO₂, EtOAc/TEA 9:1) gave [F018] as a white foam in 3.01 g, 63% yield. TLC R_{f}=0.6 (SiO₂, DCM/EtOAc 1:1). ¹H and ¹³C NMR are consistent with reported data [Mao et al 2021 ACS Chem Biol 16:76 doi:10.1021/acschembio.0c00735; Moreno et al 2022 Monatsh Chem 153:285 doi:10.1007/s00706-022-02896-x].

Synthesis of [F019] (FIG. 4): To [F018] (3.0 g, 4.51 mmol) and imidazole (0.61 g, 9.03 mmol) in DMF (40 mL) was added tert-butyldimethylchlorosilane (0.81 g, 5.42 mmol). The solution was stirred at 60° C. for 1.5 h. After cooling to rt, volatiles were evaporated. Flash column chromatography (SiO₂ hexanes/EtOAc 5:1) gave [F019] as a white foam in 1.07 g, 30% yield. TLC 2′-O-TBDMS isomer R_{f}=0.3, 3′-O-TBDMS isomer R_{f}=0.2, (SiO₂, hexanes/EtOAc 5:1). ¹H and ¹³C NMR are consistent with reported data [Mao et al 2021 ACS Chem Biol 16:76 doi:10.1021/acschembio.0c00735; Moreno et al 2022 Monatsh Chem 153:285 doi:10.1007/s00706-022-02896-x].

Synthesis of [F020] (FIG. 4): Compound [F019] (0.30 g, 0.385 mmol) and diisopropylammonium tetrazolide (0.099 g, 0.578 mmol) were dissolved in DCM (10 mL). Tetrazole (0.45 M in ACN, 1.28 mL, 0.578 mmol) and 2-cyanoethyltetraisopropylphosphorodiamidite (0.18 mL, 0.578 mmol) were added sequentially. The mixture was stirred at rt overnight under nitrogen. Volatiles were evaporated under reduced pressure. Flash column chromatography (SiO₂, hexanes/ethyl acetate 5:1 to 4:1) gave [F020] as a white foam in 0.28 g, 75% yield. TLC two isomers R_{f}=0.28, 0.26 (hexanes/EtOAc 5:1). ¹H, ¹³C, and ³¹P NMR are consistent with reported data [Mao et al 2021 ACS Chem Biol 16:76 doi:10.1021/acschembio.0c00735; Moreno et al 2022 Monatsh Chem 153:285 doi:10.1007/s00706-022-02896-x].

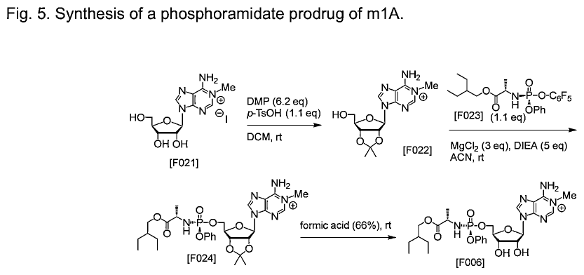

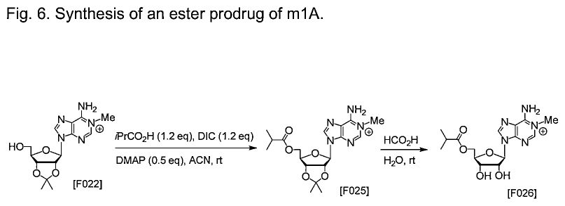

The syntheses of [F006] and [F026] are given as examples for the synthesis of m1A and m3C based prodrugs. The synthetic Schemes are provided in FIG. 5 and FIG. 6, respectively.

Synthesis of [F022] (FIG. 5): The compound can be synthesized using a similar procedure in the literature for the synthesis of a similar compound [Chen et al 2023 Org Process Res Dev doi:10.1021/acs.oprd.3c00248]. Combine [F021] (1 eq), 2,2-dimethoxypropane (DMP, 6.2 eq), p-toluenesulfonic acid (p-TsOH, 1.1 eq), and DCM (acetone also works) in a round bottom flask. Stir the mixture at rt under nitrogen until the desired conversion of starting material to product is reached as indicated by TLC. Partition the mixture between DCM and NaHCO₃ (5%) containing 5% NaPF₆ (if acetone is used as the solvent for the reaction, add NaHCO₃ (5%) containing 5% NaPF₆, evaporate the majority of acetone, add DCM, then partition). Dry the organic layer over anhydrous Na₂SO₄, filter, and concentrate the filtrate to dryness. Purify the product with flash chromatography as needed.

Synthesis of [F024] (FIG. 5): The compound can be synthesized using a similar procedure in the literature for the synthesis of a similar compound [Kumar et al 2022 Tetrahedron Lett 88:153590 doi:10.1016/j.tetlet.2021.153590; Hu et al 2022 ACS Omega 7:27516 doi:10. 1021/acsomega.2c02835; Siegel et al 2017 J Med Chem 60:1648 doi:10.1021/acs.jmedchem.6b01594]. Combine [F022] (1 eq), [F023] (1.1 eq), MgCl₂ (2 eq), DIEA (5 eq), and ACN. Stir the reaction mixture under nitrogen at rt until the desired conversion of starting material to product is reached as indicated by TLC. Evaporate ACN under reduced pressure. Partition the mixture between DCM and NaHCO₃ (5%) containing 5% NaPF₆. Dry the organic layer over anhydrous Na₂SO₄, filter, and concentrate the filtrate to dryness. Purify the product with flash chromatography as needed.

Synthesis of [F006] (FIG. 5): The compound can be synthesized using a similar procedure in the literature for the synthesis of a similar compound [Chen et al 2023 Org Process Res Dev doi:10.1021/acs.oprd.3c00248; Siegel et al 2017 J Med Chem 60:1648 doi:10.1021/acs.jmedchem.6b01594]. Combine [F024] (1 eq), and 66% formic acid in a round bottom flask at 0° C. Stir the reaction mixture at rt under nitrogen until the desired conversion of starting material to product is reached as indicated by TLC. Cool the reaction mixture to 0° C. Add EtOAc. Add 20% Na₂CO₃ containing 5% NaPF₆ until pH 7. Obtain the organic phase, dry over anhydrous Na₂SO₄, filter, and concentrate the filtrate to dryness. Purify the product with flash chromatography as needed.

Synthesis of [F025] (FIG. 6): The compound can be synthesized using a similar procedure in the literature for the synthesis of a similar compound [Chen et al 2023 Org Process Res Dev doi:10.1021/acs.oprd.3c00248]. Combine [F022] (1 eq), iPrCO₂H (1.2 eq), DMAP (0.5 eq), and ACN in a round bottom flask. Cool the flask to 5° C. Add the solution of DIC (1.2 eq) in ACN to the mixture slowly with stirring. After addition, continue to stir until the starting material disappears as indicated by TLC. Remove the DIU precipitate by filtration. Add EtOAc and 20% citric acid containing 5% NaPF₆ to the filtrate. Obtain the organic phase, dry over anhydrous Na₂SO₄, filter, and concentrate the filtrate to dryness. Purify the product with flash chromatography as needed.

Synthesis of [F026] (FIG. 6): The compound can be synthesized using a similar procedure in the literature for the synthesis of a similar compound [Chen et al 2023 Org Process Res Dev doi:10.1021/acs.oprd.3c00248]. Combine [F025] and the solution of formic acid in water (66%) in a round bottom flask. Stir the reaction mixture vigorously at rt until the starting material is consumed as indicated by TLC. Cool the reaction mixture to 0° C. Add EtOAc. Add 20% Na₂CO₃ containing 5% NaPF₆ until pH 7. Obtain the organic phase, dry over anhydrous Na₂SO₄, filter, and concentrate the filtrate to dryness. Purify the product with flash chromatography as needed.

### Procedure for the Synthesis of RNA or DNA Templates

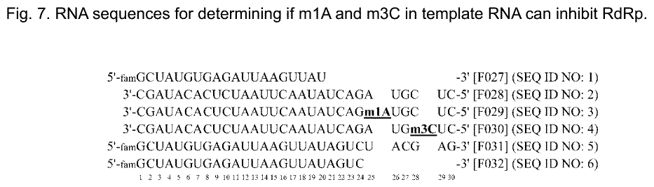

For conducting enzymatic assays to determine if the m1A or m3C modification in template RNA or DNA could inhibit a RNA or DNA polymerase, or cause mutagenesis, an RNA or DNA template containing the modification is needed. The syntheses of RNAs [F029] (SEQ ID NO: 3) and [F030] (SEQ ID NO: 4) are given as examples. The sequences are provided in FIG. 7.

The RNAs [F029] (SEQ ID NO: 3) and [F030] (SEQ ID NO: 4) were synthesized on a MerMade 6 DNA/RNA synthesizer at 1 μmol scale using standard phosphoramidite chemistry. 2′-Ac-Ac-C-succinyl-CPG was used as the solid support. Deblocking: TCA (2% in DCM), 9 sec×3. Coupling: 5′-ODMTr, 2′-OTBDMS, CE-phosphoramidites of Bz-A, Ac-C, Ac-G, U, and Cl-Ac-m1A (available from commercial sources, for the synthesis of RNA [F029], SEQ ID NO: 3) (0.1 M in ACN), Bz-m3C ([F020] for the synthesis of RNA [F030], SEQ ID NO: 4) (0.125 M in ACN), 5-(ethylthio)-1H-tetrazole (ETT, 0.25 M in ACN), 6 min×3 except for m1A (15 min×3) and m3C (12 min×3) phosphoramidites. Capping: Cap A THF/pyridine/AC₂O, Cap B Melm (16% in THF), 50 sec×3. Oxidation: I₂ (0.1 M in THF/pyridine/H₂O), 40 sec×3. Cleavage and removal of nucleobase protection groups: For RNA [F029] (SEQ ID NO: 3), the CPG was treated with the solution of NH₃ in CH₃OH (2.0 M), rt, 60 h. The supernatant was transferred to a clean centrifuge tube, and the CPG was washed with a solution of ammonia in CH₃OH (2.0 M). For [F030] (SEQ ID NO: 4), the CPG was treated with 28% NH₄OH at rt for 16 h. The supernatant was transferred to a clean centrifuge tube, and the CPG was washed with water. For both RNAs, the supernatant and the washes were combined, and volatiles were evaporated using a vacuum centrifugal evaporator. Removal of 2′-OH protecting groups: For both RNAs, the RNA was dissolved in DMSO (115 μL). The mixture was heated at 65° C. for 5 min if RNA was not dissolved completely. Triethylamine (TEA, 60 μL) was added. After mixing, TEA-3HF (75 μL) was added. The mixture was heated at 65° C. for 2.5 h. After cooling on ice, Glen-Pak RNA Quenching Buffer (1.75 mL) was added. After mixing, the quenched RNA solution was loaded on a Glen-Pak™ RNA purification cartridge that was preconditioned using ACN (0.5 mL) followed by TEAA (2 M, 1.0 mL). The cartridge was washed sequentially with the mixture of ACN and 2 M TEAA solution (1:9 v/v, pH 7.0, 1.0 mL), RNase free water (1.0 mL), TFA (2%, 1.0 mL×2, 5′-ODMTr deprotection), and deionized water (1.0 mL×2). The fully deprotected RNA was then eluted with the solution of 0.1 M NH₄HCO₃ in 30% ACN (1.0 mL). The solution was evaporated to dryness and analyzed with RP HPLC using conditions described elsewhere [Shahsavari et al 2019 Beilstein J Org Chem 15:1116 doi:10.3762/bjoc. 15.108]. All RNAs were desalted using RP HPLC or the dissolve-spin method [Apostle et al 2022 J Mass Spectrom doi:10.1002/jms.4893]. Their sequences were confirmed using MALDI MS. RNA [F029] (SEQ ID NO: 3): Calcd for C₂₈₄H₃₅₃N₁₀₇O₂₀₇P₂₉ [M-H]⁻ m/z 9474.3, found 9475.7. RNA [F030] (SEQ ID NO: 4): Calcd for C₂₈₄H₃₅₃N₁₀₇O₂₀₇P₂₉ [M-H]⁻ m/z 9474.3, found 9475.3. The neutral form of the m1A and m3C nucleobases was used for calculating the molecular mass [Fang 2021 ChemRxiv doi:10.26434/chemrxiv-2021-qkd7w].

### Procedure for Screening Compounds Using Polymerase

All compounds in this invention can be screened for suitability for treating human diseases via inhibition of DNA or RNA polymerase at the enzyme level by persons skilled in the art without excessive experimentation. The descriptions or examples provided herein are intended for illustrative purposes only and should not be construed as limiting the scope of the invention.

Determine if the m1A or m3C modifications in template RNA or DNA could inhibit a RNA or DNA polymerase: The SARS-COV-2 RdRp is used as the example. Sources of materials used in the experiment can be found in reference [Apostle et al 2023 ChemBioChem doi:10.1002/cbic.202300095]. The solution of RNA [F027] (SEQ ID NO: 1, 20 μM), RNA [F028] (SEQ ID NO: 2, 20 μM), Tris-HCl (10 mM) and KCl (100 mM) with indicated final concentration was prepared. The solution was heated at 94° C. for 5 min, and then cooled to rt slowly to give the duplex solution of [F027]-[F028] (SEQ ID NOs: 1 and 2). The RNA extension reaction solution (35 μL) containing SARS-COV-2 RdRp (1.2 M), Tris-HCl (20 mM, pH 8), KCl (50 mM), MgCl₂ (6 mM), DTT (1 mM), RNase inhibitor (1.12 U/μL), ATP (0.5 mM), CTP (0.5 mM), GTP (0.5 mM), UTP (0.5 mM) and the RNA duplex of [F027]-[F028] (SEQ ID NOs: 1 and 2, 1.5 μM) with indicated final concentration was prepared. The RNA duplex solution was added last, and upon its addition, the mixture was immediately agitated by a brief vortex and spin. The solution was equally aliquoted into seven PCR tubes. The tubes were immediately placed into a PCR instrument, and heated at 37° C. for 0 min (not placed in PCR instrument), 5 min, 10 min, 20 min, 40 min, 2 h and 6 h, respectively. The reactions were quenched with an EDTA solution (final concentration 50 μM) followed by RNA loading dye (final concentration 1×). The samples were immediately stored at −80° C. until analysis by gel electrophoresis. A portion of the samples (equivalent to 0.0331 μg [F028], SEQ ID NO: 2) was analyzed with electrophoresis using 10% Mini-PROTEAN® TBE-Urea Gel in a Mini-PROTEAN Tetra Cell at 200 V for 32 min. The gel was first imaged without staining using UVP GelDoc-IT Imaging System 2UV Transilluminator at 302 nm. The same gel was then stained with GelRed (final concentration 300×) for 32 min and imaged again at 302 nm. The primer extension reactions involving templates [F029] (SEQ ID NO: 3) and [F030] (SEQ ID NO: 4), and their analyses were performed under the same conditions except that in the case of [F029] (SEQ ID NO: 3) more samples (equivalent to 0.0946 μg [F029], SEQ ID NO: 3) were used for gel electrophoresis analysis.

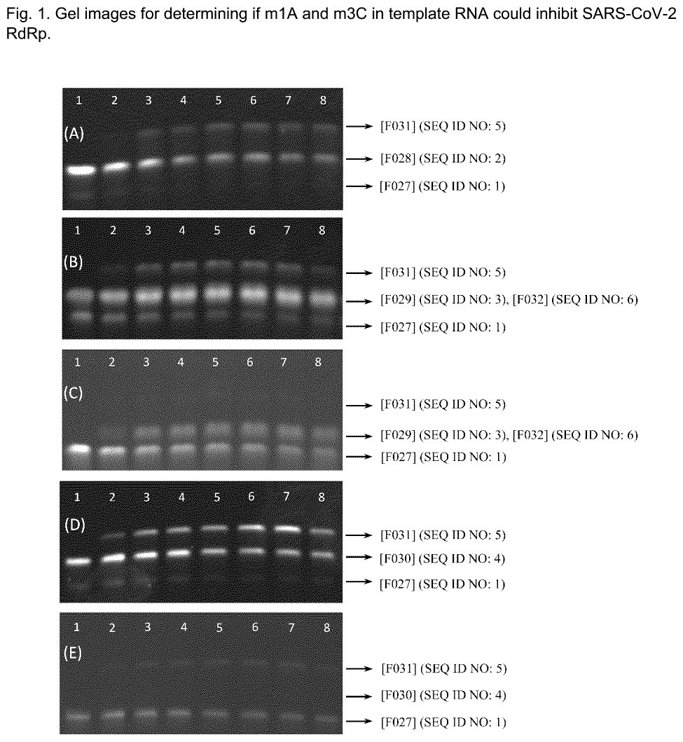

The results of the primer extension reaction is shown in FIG. 1, gel images for determining if m1A and m3C in template RNA could inhibit SARS-COV-2 RdRp. The 20-mer primer [F027] (SEQ ID NO: 1) of the duplex of [F027] (SEQ ID NO: 1) and the 30-mer template [F028] (SEQ ID NO: 2) was extended in the presence of the four canonical NTPs as described above. RNAs corresponding to the bands on the gel are indicated at the right side of the images. The primer [F027] (SEQ ID NO: 1) and its extension products [F031] (SEQ ID NO: 5) and [F032] (SEQ ID NO: 6) have a 5′-FAM. The templates [F028] (SEQ ID NO: 2), [F029] (SEQ ID NO: 3) and [F030] (SEQ ID NO: 4) do not have a 5′-FAM. Lanes 1-8 are from the mixture of reactions quenched at 0 min, 5 min, 10 min, 20 min, 40 min, 2 h and 6 h, respectively. (A) Unmodified [F028] (SEQ ID NO: 2) was used as the template. Image of stained gel. Primer [F027] (SEQ ID NO: 1) was converted to [F031] (SEQ ID NO: 5) in ˜20 min. (B) RNA [F029] (SEQ ID NO: 3) containing m1A was used as the template. Image of stained gel. Primer [F027] (SEQ ID NO: 1) remained after 6 h. Small amount [F031] (SEQ ID NO: 5). Truncated extension product [F032] (SEQ ID NO: 6) overlapped with [F029] (SEQ ID NO: 3). The data indicate that m1A in template RNA inhibits RdRp. (F) Image of unstained gel of (E). Only RNAs [F027] (SEQ ID NO: 1), [F031] (SEQ ID NO: 5) and [F032] (SEQ ID NO: 6), which contain FAM, are visible. RNA template [F029] (SEQ ID NO: 3) is invisible. The data indicate the amount of [F031] (SEQ ID NO: 5) is very little, and the majority of RNA corresponding to the location of [F029] (SEQ ID NO: 3) and [F032] (SEQ ID NO: 6) is [F032] (SEQ ID NO: 6) because [F029] (SEQ ID NO: 3) does not contain FAM and is invisible. This further confirms that m1A inhibits RdRp. (G) RNA [F030] (SEQ ID NO: 4) containing m3C was used as the template. Image of stained gel. Primer [F027] (SEQ ID NO: 1) was converted to [F031] (SEQ ID NO: 5) in ˜2 h. The data indicate that the RdRp can tolerate m3C. (H) Image of unstained gel of (G). Only RNAs [F027] (SEQ ID NO: 1) and [F031] (SEQ ID NO: 5), which contain FAM, are visible. RNA [F030] (SEQ ID NO: 4) is invisible. No band is visible between bands of [F027] (SEQ ID NO: 1) and [F031] (SEQ ID NO: 5) indicating that no truncated RNAs were formed. Overall, the data indicate that m1A in template RNA inhibits SARS-COV-2 RdRp, while m3C in template RNA can be tolerated by the RdRp.

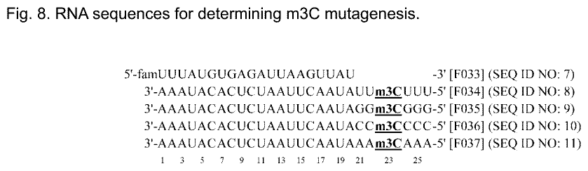

Determine if the m3C modification in template RNA or DNA could cause mutagenesis: The SARS-COV-2 RdRp is used as the example. Synthesize RNAs [F033-F037] (SEQ ID NOs: 7-11, sequences are in FIG. 8) using the procedure described for the synthesis of RNAs [F029-F030] (SEQ ID NOs: 3-4). Carry out primer extension reactions involving primer-template duplex [F033]-[F034] (SEQ ID NOs: 7 and 8), SARS-COV-2 RdRp, and ATP (no CTP, GTP or UTP) under similar conditions described earlier in this patent application. Perform gel electrophoresis, and determine if A can be incorporated into RNA across m3C in the template [F034] (SEQ ID NO: 8). Similarly, determine if C, G, and U can be incorporated in RNA across m3C in the template using primer-template duplexes [F033]-[F035] (SEQ ID NOs: 7 and 9), [F033]-[F036] (SEQ ID NOs: 7 and 10), and [F033]-[F037] (SEQ ID NOs: 7 and 11), respectively. Determine the relative rates for the incorporation of A, C, G and U across m3C in the template by analyzing the gel images using gel densitometry. The results give quantitative data regarding mutagenesis caused by the m3C modification. Mutagenesis caused be the m1A modification, if it does not inhibit a polymerase from a virus or cancer cell, can be determined similarly.

Determine if the m1A or m3C modification could be incorporated onto RNA or DNA by a polymerase via m1ATP or m3CTP, respectively: The SARS-COV-2 RdRp with m1ATP is used as the example. Sources of materials used in the experiment can be found in reference [Apostle et al Preprints doi:10.20944/preprints202311.1268.v1]. RNA duplex [F038]-[F039] (SEQ ID NOs: 12 and 13, FIG. 2) was prepared by annealing the two RNAs using the following procedure. The solution of RNAs [F038] (SEQ ID NO: 12, 20 μM, all concentrations are final) and [F039] (SEQ ID NO: 13, 20 μM) in Tris-HCl (10 mM), and KCI (100 mM) was heated at 94° C. for 5 min. The solution was then cooled to RT slowly giving the [F038]-[F039] (SEQ ID NOs: 12 and 13) duplex. The primer extension experiment: To five PCR tubes containing MgCl₂ (5 mM, all concentrations are final), NaCl (50 mM), HEPES (pH 7.5, 20 mM), RNA duplex (1.5 μM), and SARS-COV-2 RdRP (1 μM) were added, respectively, the following NTPs with each NTP having a final concentration of 0.5 mM. The five tubes corresponded to the experiments for lanes 2 to 6 in FIG. 2. Tube for lane 2: ATP, CTP, GTP, and UTP. Tube for lane 3: m¹ATP, CTP, GTP, and UTP. Tube for lane 4: m¹ATP only, after incubating the mixture at 37° C. for 15 min., added ATP, CTP, GTP, and UTP. Tube for lane 5: CTP, GTP, and UTP. Tube for lane 6: ATP, m¹ATP, CTP, GTP, and UTP. The final volumes in each tube were 10 μL. The five tubes were incubated at 37° C. for a total of 120 min. The tubes were cooled on ice immediately following incubation, whereupon 5 μL of a 150 μM EDTA solution and 15 μL of 2× RNA loading dye were added sequentially achieving a final volume of 30 μL. To another tube containing only the RNA duplex in 10 μL nuclease free water (1.5 M, corresponding to the experiment of lane 1 in FIG. 2) were also added the EDTA and loading dye solutions (same quantities as above, final volume 30 μL). Aliquots of 10 μL solution from each of the six tubes were loaded onto a 10% PAGE-Urea gel. Electrophoresis was run at 200 V for 35 min. The gel was stained with GelRed (final concentration 300×) for 35 min., and then rinsed with DI water for 5 min. Gel image (FIG. 2) was obtained using a UVP GelDoc-IT Imaging System 2UV Transilluminator at 302 nm.

The results of the primer extension reaction is shown in FIG. 2, gel image for determining if m1A could be incorporated onto RNA by SARS-COV-2 RdRp via m1ATP. X in [F040] (SEQ ID NO: 14) is the nucleoside A or m1A depending on the NTP used. As described above, the mixture of RdRp, primer-template duplex [F038]-[F039] (SEQ ID NOs: 12 and 13) and NTPs was incubated at 37° C. for 2 h. The reaction products were analyzed with PAGE. The image was taken after staining with GelRed. Lane 1: RNA duplex only. Lane 2: ATP, CTP, GTP and UTP, no m1ATP. Lane 3: m1ATP, CTP, GTP and UTP, no ATP. Result indicates that m1A can be incorporated into RNA via m1ATP. Lane 4: m1ATP only for 15 min, then ATP, CTP, GTP and UTP. Results indicate m1ATP does not inhibit RdRp. Lane 5: CTP, GTP, UTP, no ATP, no m1ATP. Results indicate that ATP and m1ATP in other lanes were needed to reach 30-mer RNA. Results also indicate that one or more of the nucleotides in CTP, GTP or UTP can be incorporated into RNA across U in the template although with low efficiency as indicated by close to complete disappearance of primer, appearance of partially extended primer and small amount of fully extended primer. The latter may also be partially extended 28-mer. This agrees with the low fidelity of RdRp. Lane 6: ATP, m1ATP, CTP, GTP and UTP. Overall, the results indicate that m1A can be incorporated into RNA by SARS-COV-2 RdRp via m1ATP.

### Procedure for Screening Compounds Using Replicon

All compounds in this invention can be screened for suitability for treating human diseases via inhibition of DNA or RNA polymerase using replicon by persons skilled in the art without excessive experimentation. The descriptions or examples provided herein are intended for illustrative purpose only and should not be construed as limiting the scope of the invention.

Screening m1A and m3C based prodrugs using the SARS-COV-2 replicon and the Huh-7.5 cell line reported in the literature is used as the example [Ricardo-Lax et al 2021 Science 374:1099 doi:10. 1126/science.abj8430]. Other replicons for different viruses are also available, and the screening procedure is similar [Khan et al 2020 Front Cell Infect Microbiol 10:325 doi:10.3389/fcimb.2020.00325; Lin et al 2023 Microbiol Spectr 11: e0485422 doi:10.1128/spectrum.04854-22; Zhang et al 2022 J Med Virol 94:2438 doi: 10.1002/jmv.27650]. The SARS-COV-2 replicon can be constructed according to literature procedure [Ricardo-Lax et al 2021 Science 374:1099 doi:10.1126/science.abj8430]. The advantages of using the replicon in place of real virus include non-contagiousness due to the lack of the spike protein gene by the replicon compared with the real virus, and the ease of screening due to the inclusion of the Gaussia luciferase gene in the replicon. Procedure: Trypsinize Huh-7.5 (or BHK-21) cell and wash the cell with ice-cold PBS buffer. Resuspend the cell in PBS at a concentration of 1.5×10⁷ cells/mL. Mix 5 μg SARS-CoV-2 replicon RNA, 2 μg SARS-CoV-2 N mRNA, and 0.4 mL cell suspension in a 2 mm cuvette. Pulse the mixture electrically using a BTX ElectroSquare Porator ECM 830. Incubate the cells at rt for 10 min. Plate 15-30K cells in 100 UL media per well into 96-well plates containing 100 μL of dilutions of a prodrug or control material (blank control, known drug such as Remdesivir) at 2× desired final concentrations. Harvest cell supernatants cumulatively at various timepoints or at 24 h post-electroporation. Measure luciferase signal using a luciferase assay system following the protocol provided by instrument manufacturer. Measure cell viability using a cell viability assay system following the protocol provided by instrument manufacturer. From the signals, determine IC₅₀ values and cytotoxicity profiles.

### Procedure for Screening Compounds Using Virus

All compounds in this invention can be screened for suitability for treating human diseases via inhibition of DNA or RNA polymerase using live virus by persons skilled in the art without excessive experimentation following well documented procedures such as those described in this publication [Chun et al 2019 Pat No U.S. Pat. No. 10,251,898B2]. The descriptions or examples provided herein are intended for illustrative purpose only and should not be construed as limiting the scope of the invention.

The SARS-CoV-2 virus with Huh-7 cell is used as the example. Seed Huh-7 cells in 96 well plates. Prepare solutions of prodrugs in suitable media at eight to ten different concentrations by diluting an stock solution in 3-fold serial increments. Transfer the solution of each of the concentrations to three wells containing the pre-seeded Huh-7 cell monolayers in the 96 well plates. Transfer the plates into BSL-4 containments. Add appropriately diluted SARS-CoV-2 virus in cell culture media to the test plates containing the cells and serially diluted compounds. Eash plate should include three wells that serve as 0% and 100% virus inhibition control, respectively. Incubate the plates for 3 to 4 days in a tissue culture incubator. Remove media from the infected cells. Quantify viral RNA by RT-qPCR using a portion of the media. Calculate the percentage of inhibition for each tested concentration relative to the 0% and 100% inhibition controls. Determine the EC₅₀ value for each prodrug by non-linear regression as the effective concentration of prodrug that inhibited virus replication by 50%.

### Procedure for Screening Compounds Using Cancer Cell

All compounds in this invention can be screened for suitability for treating human diseases via inhibition of DNA or RNA polymerase using cancer cells following procedures as reviewed in this reference [Kitaeva et al 2020 Front Bioeng Biotechnol 8: doi:10.3389/fbioe.2020.00322] by persons skilled in the art without excessive experimentation, or by established screening programs in various organizations such as the US National Cancer Institute. Additional studies needed for anticancer drug development are also standard. The descriptions provided herein are intended for illustrative purpose only and should not be construed as limiting the scope of the invention.

This example anticancer screening method is based on existing descriptions [Chang et al 2015 Pat No U.S. Pat. No. 9,095,599B2]. Test compounds in leukemic cell lines for anticancer efficacy using the MTS assay reagents from Promega (CellTiter96 Aqueous One solution proliferation assay) following manufacturer provided protocol.

This example anticancer screening method is based on existing descriptions [Wagner 2018 Pat Appl No US2018/0258131A1]. Grow CCRF-CEM cells in a medium containing RPMI 1640 supplemented with 10% heat-inactivated fetal bovine serum, penicillin (124 units/mL of medium) and streptomycin (0.125 mg/mL of medium). Make a stock solution of 10⁵ cells using Trypan Blue Dye Exclusion Method as a means of cell counting. Prepare solutions of the compounds to be tested with the concentration of 250, 200, 100, 1, 0.1, 0.01 and 0.001 μM. Mix the cell stock (50 μL, 5×10⁴ cells) with 50 μL of each of the compounds in triplicate in a 96-well plate. Incubate the mixture at 37° C. in a 10% CO₂ 90% air environment for 48 hours. At the same time, carry out a negative control experiment where the cells are incubated in drug-free medium. Determine cell viability by adding 20 μL MTS reagent and incubating the cells for 4 hours, and then measure absorbance at 490 nm. Determine IC₅₀ values.

1. A compound of structural Formula (I) or (II) 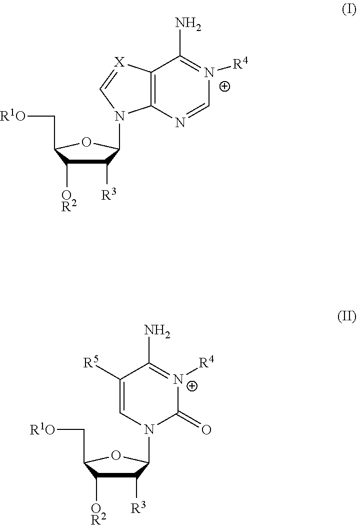
or a pharmaceutically acceptable salt, hydrate, ester, amide, or formulation thereof, wherein, R¹ is selected from the group consisting of items in subgroups (a-c):
(a) —(C═O) R^{1a},
wherein R^{1a} is (C₃-C₂₀)alkyl, (C₂-C₂₀)alkenyl, (C₂-C₂₀)alkynyl, (C₄-C₈) carbocyclylalkyl, (C₆-C₂₀) optionally substituted aryl, (C₆-C₂₀) optionally substituted heteroaryl, or (C₆-C₂₀)aryl(C₁-C₈)alkyl;
(b) —(C═O)OR^{1b}, —C(C═O)NR^{1b}R^{1c}, —C(C═O)SR^{1b}, —S(O)R^{1b}, —S(O)₂R^{1b}, —S(O)(OR^{1b}), —S(O)₂(OR^{1b}), and —SO₂NR^{1b}R^{1c};
wherein each R^{1b} and R^{1c} is independently H, (C₁-C₂₀)alkyl, (C₂-C₂₀) alkenyl, (C₂-C₂₀)alkynyl, (C₄-C₈)carbocyclylalkyl, (C₆-C₂₀) optionally substituted aryl, (C₆-C₂₀) optionally substituted heteroaryl, or (C₆-C₂₀)aryl (C₁-C₈)alkyl; or R^{1b} and R^{1c} taken together with a nitrogen to which they are both attached form a 3 to 7 membered heterocyclic ring wherein any one carbon atom of said heterocyclic ring can optionally be replaced with —O—, —S— or —NR^{1d}; wherein
each R^{1d} is independently selected from the group consisting of H, (C₁-C₂₀)alkyl, (C₂-C₂₀)alkenyl, (C₂-C₂₀)alkynyl, (C₆-C₂₀)aryl, (C₆-C₂₀)aryl (C₁-C₈)alkyl, (C₄-C₈)carbocyclylalkyl, —C(═O)R^{1e}, —C(═O)OR^{1e}, —C(═O)NR^{1e}₂, —C(═O)SR^{1e}, —S(O)R^{1e}, —S(O)₂R^{1e}, —S(O)(OR^{1e}), —S(O)2(OR^{1e}), and —SO₂NR^{1e}₂; wherein
each R^{1e} is independently selected from the group consisting of H, (C₁-C₂₀)alkyl, (C₁-C₂₀) substituted alkyl, (C₂-C₂₀)alkenyl, (C₂-C₂₀) substituted alkenyl, (C₂-C₂₀)alkynyl, (C₂-C₂₀) substituted alkynyl, (C₆-C₂₀)aryl, (C₂-C₂₀) substituted aryl, (C₂-C₂₀)heterocyclyl, (C₂-C₂₀) substituted heterocyclyl, (C₆-C₂₀)aryl(C₁-C₈)alkyl, and substituted (C₆-C₂₀)aryl (C₁-C₈)alkyl;
wherein each (C₁-C₂₀)alkyl, (C₂-C₂₀)alkenyl, (C₂-C₂₀)alkynyl, and (C₆-C₂₀)aryl (C₁-C₈)alkyl of each R^{1a}, R^{1b}, R^{1c}, R^{1d} and R^{1e} is, independently, optionally substituted with one or more substituents selected from the group consisting of halo, hydroxy, CN, N₃, N(R^{1d})₂ and OR^{1d-}; and wherein one or more of the non-terminal carbon atoms of each said (C₁-C₂₀)alkyl is optionally replaced with —O—, —S—, or —NR^{1d}—; and
(c) Formulas (III-V) 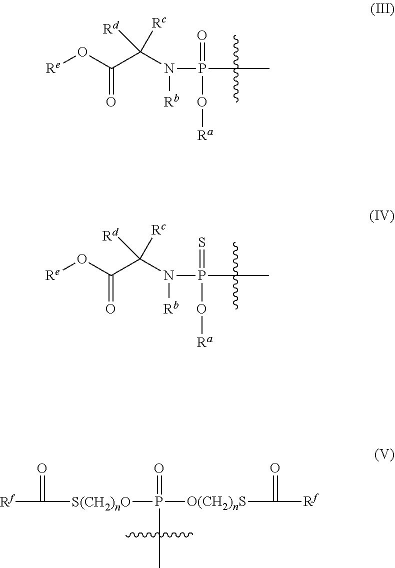
wherein
R^{a} is selected from the group consisting of phenyl, 1-naphthyl, 2-naphthyl, quinolinyl, and isoquinolinyl;
R^{b} is selected from the group consisting of H, Me, —(CH₂)₂OMe, —(CH₂)₂O-substituted Me, —(CH₂)₂OPh, and —(CH₂)₂O-substituted Ph;
R^{c} and R^{d} are each independently selected from the group consisting of H, (C₁-C₆)alkyl, allyl, propargyl, and benzyl;
R^{e} is selected from the group consisting of H, (C₁-C₈)alkyl, benzyl, allyl, propargyl, (C₃-C₆)cycloalkyl, and —CH₂—(C₃-C₆)cycloalkyl;
R^{f} is selected from the group consisting of (C₁-C₈)alkyl, —O—(C₁-C₈)alkyl, benzyl, —O-benzyl, allyl, —O-allyl, propargyl, —O-propargyl, —CH₂—(C₃-C₆) cycloalkyl, —O—CH₂—(C₃-C₆)cycloalkyl, and CF₃; and
n is an integer selected from the group consisting of 1, 2, 3, and 4;
R² is selected from the group consisting of H, Me, —(C═O)R^{1b}, —(C═O)OR^{1b}, —C(C═O)NR^{1b}R^{1c}, —C(C═O)SR^{1b}, —S(O)R^{1b}, —S(O)₂R^{1b}, —S(O)(OR^{1b}), —S(O)₂(OR^{1b}), and —SO₂NR^{1b}R^{1c};
R³ is selected from the group consisting of H, F, O(CH₂)₂OH, O(CH₂)₂OMe, O(CH₂)₂NH₂, O(CH₂)₂NHMe, O(CH₂)₂NMe₂, ONH₂, and OR²;
R⁴ is selected from the group consisting of Me and Et;
R⁵ is selected from the group consisting of H and Me; and
X is selected from the group consisting of N, and CH.

2. The compound of claim 1, or a pharmaceutically acceptable salt, hydrate, ester, amide, or formulation thereof, wherein said structural Formula is (I).

3. The compound of claim 1, or a pharmaceutically acceptable salt, hydrate, ester, amide, or formulation thereof, wherein said structural Formula is (II).

4. The compound of claim 1, or a pharmaceutically acceptable salt, hydrate, ester, amide, or formulation thereof, wherein said R¹ is —(C═O) R^{1a} with R^{1a} being (C₃-C₂₀)alkyl.

5. The compound of claim 1, or a pharmaceutically acceptable salt, hydrate, ester, amide, or formulation thereof, wherein said R¹ is —(C═O) OR^{1b} with R^{1b} being (C₁-C₂₀)alkyl.

6. The compound of claim 1, a pharmaceutically acceptable salt, hydrate, ester, amide, or formulation thereof, wherein said R¹ is selected from the group consisting of Formulas (III-IV).

7. The compound of claim 1, or a pharmaceutically acceptable salt, hydrate, ester, amide, or formulation thereof, wherein said R¹ is Formula (V).

8. The compound of claim 1, or a pharmaceutically acceptable salt, hydrate, ester, amide, or formulation thereof, wherein said R¹ is selected from the group consisting of Formulas (III-IV), R² is H, and R³ is selected from the group consisting of H and OH.

9. The compound of claim 1, or a pharmaceutically acceptable salt, hydrate, ester, amide, or formulation thereof, wherein said R¹ is Formula (V), R² is H, and R³ is selected from the group consisting of H and OH.

10. The compound of claim 1, or a pharmaceutically acceptable salt, hydrate, ester, amide, or formulation thereof, wherein said R¹ is selected from the group consisting of Formulas (III-IV), R² is H, R³ is selected from the group consisting of H and OH, and R⁴ is Me.

11. The compound of claim 1, or a pharmaceutically acceptable salt, hydrate, ester, amide, or formulation thereof, wherein said R¹ is Formula (V), R² is H, R³ is selected from the group consisting of H and OH, and R⁴ is Me.

12. The compound of claim 1, or a pharmaceutically acceptable salt, hydrate, ester, amide, or formulation thereof, wherein said R¹ is selected from the group consisting of Formulas (III-IV); R^{a} is phenyl; R^{b} is selected from the group consisting of H and Me; R^{e} and R^{d} are each independently selected from the group consisting of H and Me; R^{e} is selected from the group consisting of Me, Et and isopropyl; R² is H; R³ is selected from the group consisting of H and OH; and R⁴ is Me.

13. The compound of claim 1, or a pharmaceutically acceptable salt, hydrate, ester, amide, or formulation thereof, wherein said R¹ is Formula (V); R^{f} is (C₁-C₈)alkyl; n is an integer selected from the group consisting of 2, 3, and 4; R² is H; R³ is selected from the group consisting of H and OH; and R₄ is Me.

14. The compound of claim 1, or a pharmaceutically acceptable salt, hydrate, ester, amide, or formulation thereof, wherein said structural Formula (I) is selected from the group consisting of

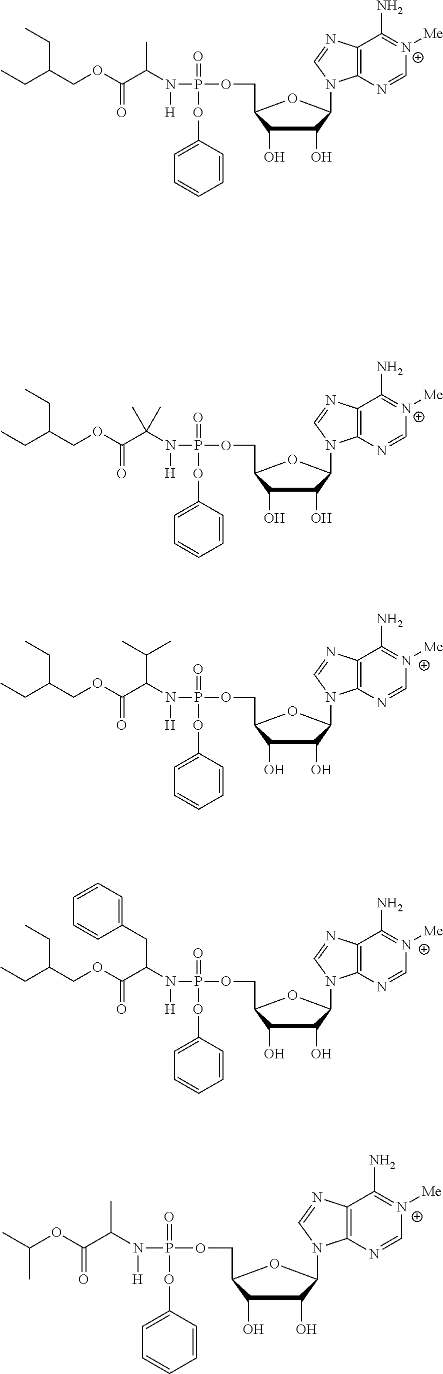

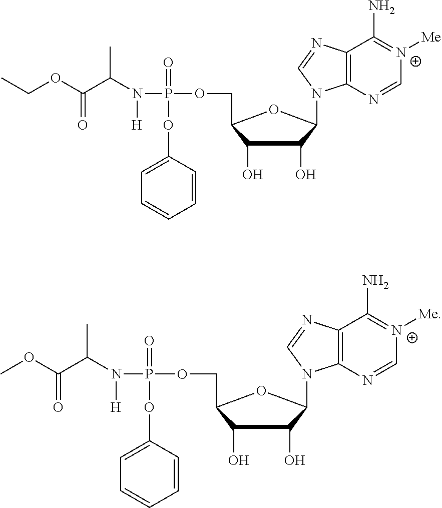

15. The compound of claim 1, or a pharmaceutically acceptable salt, hydrate, ester, amide, or formulation thereof, wherein said structural Formula (II) is selected from the group consisting of

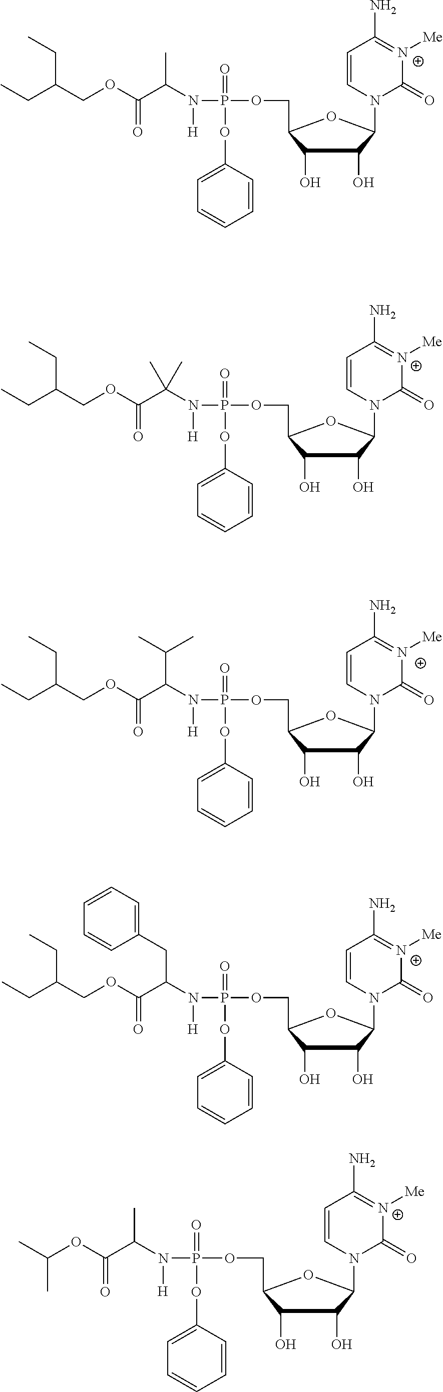

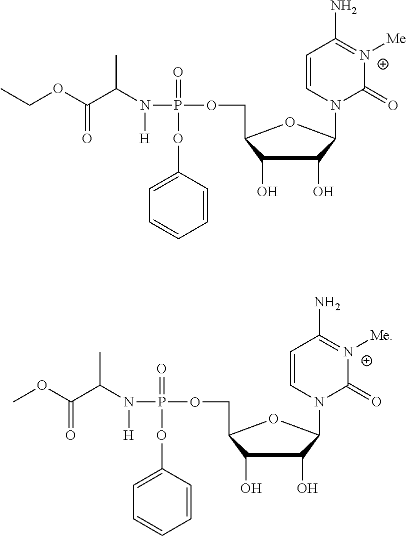

16. A method of preventing or treating cancer or infectious disease in a human involving the use of a compound with Formula (I) or (II): 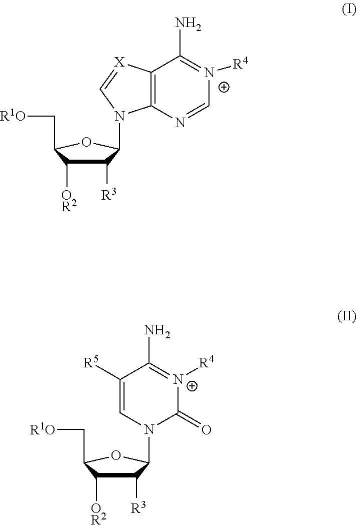
or a pharmaceutically acceptable salt, hydrate, ester, amide, or formulation thereof, wherein,
R¹ is selected from the group consisting of items in subgroups (a-c):
(a) —(C═O)R^{1a},
wherein R^{1a} is (C₃-C₂₀)alkyl, (C₂-C₂₀)alkenyl, (C₂-C₂₀)alkynyl, (C₄-C₈)carbocyclylalkyl, (C₆-C₂₀) optionally substituted aryl, (C₆-C₂₀) optionally substituted heteroaryl, or (C₆-C₂₀)aryl (C₁-C₈)alkyl;
(b) —(C═O)OR^{1b}, —C(C═O)NR^{1b}R^{1c}, —C(C═O)SR^{1b}, —S(O)R^{1b}, —S(O)₂R^{1b}, —S(O)(OR^{1b}), —S(O)₂(OR^{1b}), and —SO₂NR^{1b}R^{1c};
wherein each R^{1b} and R^{1c} is independently H, (C₁-C₂₀)alkyl, (C₂-C₂₀)alkenyl, (C₂-C₂₀)alkynyl, (C₄-C₈)carbocyclylalkyl, (C₆-C₂₀) optionally substituted aryl, (C₆-C₂₀) optionally substituted heteroaryl, or (C₆-C₂₀)aryl (C₁-C₈)alkyl; or R^{1b} and R^{1c} taken together with a nitrogen to which they are both attached form a 3 to 7 membered heterocyclic ring wherein any one carbon atom of said heterocyclic ring can optionally be replaced with —O—, —S— or —NR^{1d}; wherein
each R^{1d} is independently selected from the group consisting of H, (C₁-C₂₀)alkyl, (C₂-C₂₀)alkenyl, (C₂-C₂₀)alkynyl, (C₆-C₂₀)aryl, (C₆-C₂₀)aryl (C₁-C₈)alkyl, (C₄-C₈)carbocyclylalkyl, —C(═O)R^{1e}, —C(═O)OR^{1e}, —C(═O)NR^{1a}₂, —C(═O)SR^{1e}, —S(O)R^{1e}, —S(O)₂R^{1e}, —S(O)(OR^{1e}), —S(O)₂(OR^{1e}), and —SO₂NR^{1e}₂; wherein each R^{1e} is independently selected from the group consisting of H, (C₁-C₂₀)alkyl, (C₁-C₂₀) substituted alkyl, (C₂-C₂₀)alkenyl, (C₂-C₂₀) substituted alkenyl, (C₂-C₂₀)alkynyl, (C₂-C₂₀) substituted alkynyl, (C₆-C₂₀)aryl, (C₂-C₂₀) substituted aryl, (C₂-C₂₀)heterocyclyl, (C₂-C₂₀) substituted heterocyclyl, (C₆-C₂₀)aryl (C₁-C₈)alkyl, and substituted (C₆-C₂₀) aryl (C₁-C₈)alkyl;
wherein each (C₁-C₂₀)alkyl, (C₂-C₂₀)alkenyl, (C₂-C₂₀)alkynyl, and (C₆-C₂₀)aryl (C₁-C₈)alkyl of each R^{1a}, R^{1b}, R^{1c}, R^{1d} and R^{1e} is, independently, optionally substituted with one or more substituents selected from the group consisting of halo, hydroxy, CN, N₃, N(R^{1d})₂ and OR^{1d}; and wherein one or more of the non-terminal carbon atoms of each said (C₁-C₂₀)alkyl is optionally replaced with —O—, —S—, or —NR^{1d}—; and
(c) Formulas (III-V) 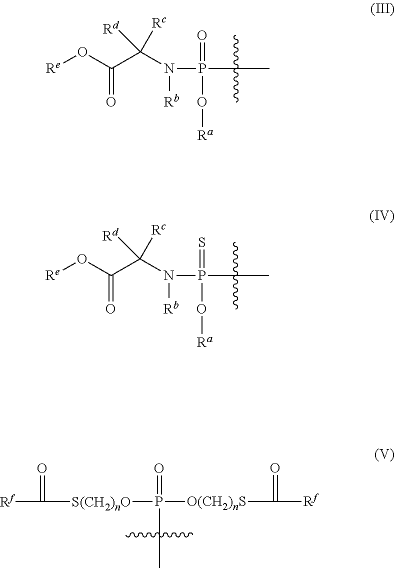
wherein
R^{a} is selected from the group consisting of phenyl, 1-naphthyl, 2-naphthyl, quinolinyl, and isoquinolinyl;
R^{b} is selected from the group consisting of H, Me, —(CH₂)₂OMe, —(CH₂)₂O-substituted Me, —(CH₂)₂OPh, and —(CH₂)₂O-substituted Ph;
R^{c} and R^{d} are each independently selected from the group consisting of H, (C₁-C₆)alkyl, allyl, propargyl, and benzyl;
R^{e} is selected from the group consisting of H, (C₁-C₈)alkyl, benzyl, allyl, propargyl, (C₃-C₆)cycloalkyl, and —CH₂—(C₃-C₆)cycloalkyl;
R^{f} is selected from the group consisting of (C₁-C₈)alkyl, —O—(C₁-C₈)alkyl, benzyl, —O-benzyl, allyl, —O-allyl, propargyl, —O—propargyl, —CH₂—(C₃-C₆)cycloalkyl, —O—CH₂—(C₃-C₆)cycloalkyl, and CF₃; and
n is an integer selected from the group consisting of 1, 2, 3, and 4;
R² is selected from the group consisting of H, Me, —(C═O)R^{1b}, —(C═O)OR^{1b}, —C(C═O)NR^{1b}R^{1c}, —C(C═O)SR^{1b}, —S(O)R^{1b}, —S(O)₂R^{1b}, —S(O)(OR^{1b}), —S(O)₂(OR^{1b}), and —SO₂NR^{1b}R^{1c};
R³ is selected from the group consisting of H, F, O(CH₂)₂OH, O(CH₂)₂OMe, O(CH₂)₂NH₂, O(CH₂)₂NHMe, O(CH₂)₂NMe₂, ONH₂, and OR₂;
R⁴ is selected from the group consisting of Me and Et;
R⁵ is selected from the group consisting of H and Me; and
X is selected from the group consisting of N, and CH.

17. The method of claim 16 wherein said R¹ is selected from items in subgroup (c).

18. The method of claim 16 wherein said Formula (I) or (II) is selected from the group consisting of

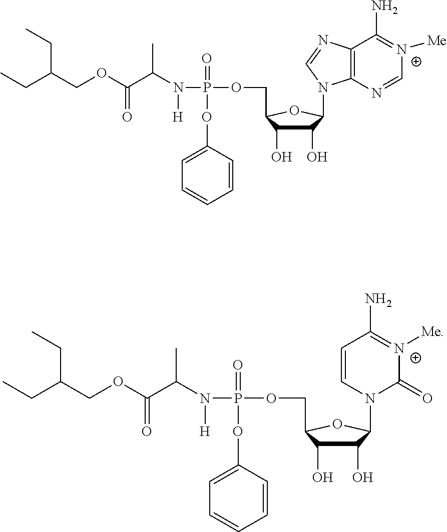

19. The method of claim 16 for preventing or treating cancer.

20. The method of claim 16 wherein said infectious disease is caused by severe acute respiratory syndrome coronavirus 2, and severe acute respiratory syndrome coronavirus.

21. The method of claim 16 wherein said infectious disease is caused by human immunodeficiency virus.

22. The method of claim 16 wherein said infectious disease is caused by filoviruses.

23. The method of claim 16 wherein said infectious disease is caused by influenza viruses.

24. The method of claim 16 wherein said infectious disease is caused by common cold viruses.

25. The method of claim 16 wherein said infectious disease is caused by hepatitis C viruses.

26. The method of claim 16 wherein said infectious disease is caused by bacteria or fungi.

27. The method of claim 16 involving the use of a combination of compounds with Formulas (I) and (II); a combination of compounds with Formulas (I) and (II), and one or more other therapeutic agents; or a combination of a compound with Formula (I) or (II), and one or more other therapeutic agents.


SEQUENCE LISTING (14 sequences)

SEQ ID NO: 1 - RNA, 20 nt, synthetic construct
  source 1..20: mol_type = other RNA
  misc_feature <1: note = 5'-FAM
gctatgtgagattaagttat

SEQ ID NO: 2 - RNA, 30 nt, synthetic construct
  source 1..30: mol_type = other RNA
ctcgtagactataacttaatctcacatagc

SEQ ID NO: 3 - RNA, 30 nt, synthetic construct; identical to SEQ ID NO: 2
  source 1..30: mol_type = other RNA
  modified_base 6: mod_base = m1a

SEQ ID NO: 4 - RNA, 30 nt, synthetic construct; identical to SEQ ID NO: 2
  source 1..30: mol_type = other RNA
  modified_base 3: mod_base = m3c

SEQ ID NO: 5 - RNA, 30 nt, synthetic construct
  source 1..30: mol_type = other RNA
  misc_feature <1: note = 5'-FAM
gctatgtgagattaagttatagtctacgag

SEQ ID NO: 6 - RNA, 24 nt, synthetic construct
  source 1..24: mol_type = other RNA
  misc_feature <1: note = 5'-FAM
gctatgtgagattaagttatagtc

SEQ ID NO: 7 - RNA, 20 nt, synthetic construct
  source 1..20: mol_type = other RNA
  misc_feature <1: note = 5'-FAM
tttatgtgagattaagttat

SEQ ID NO: 8 - RNA, 26 nt, synthetic construct
  source 1..26: mol_type = other RNA
  modified_base 4: mod_base = m3c
tttcttataacttaatctcacataaa

SEQ ID NO: 9 - RNA, 26 nt, synthetic construct
  source 1..26: mol_type = other RNA
  modified_base 4: mod_base = m3c
gggcggataacttaatctcacataaa

SEQ ID NO: 10 - RNA, 26 nt, synthetic construct
  source 1..26: mol_type = other RNA
  modified_base 4: mod_base = m3c
ccccccataacttaatctcacataaa

SEQ ID NO: 11 - RNA, 26 nt, synthetic construct
  source 1..26: mol_type = other RNA
  modified_base 4: mod_base = m3c
aaacaaataacttaatctcacataaa

SEQ ID NO: 12 - RNA, 20 nt, synthetic construct
  source 1..20: mol_type = other RNA
  misc_feature <1: note = 5'-FAM
gctatgtgagattaagttat

SEQ ID NO: 13 - RNA, 30 nt, synthetic construct; identical to SEQ ID NO: 2
  source 1..30: mol_type = other RNA

SEQ ID NO: 14 - RNA, 30 nt, synthetic construct
  source 1..30: mol_type = other RNA
  misc_feature <1: note = 5'-FAM
  variation 21: note = a or m1a
  variation 26: note = a or m1a
  variation 29: note = a or m1a
gctatgtgagattaagttatngtctncgng